SEQUENCE LISTING (90 sequences)

SEQ ID NO: 1 - DNA, 1603 bp, M. musculus
  misc_feature (1)...(1603): n = A,T,C or G
ttgggaaaaaaagccaagtagtcataaagaatttatgaancaattcctgggattgtttatatttgttacaaacaaatttatatgtttgttagtcagtaatgtataagtgggattttaaagcatgattatcttgaatttttaacaaaaaacatgtagtgctttttaaatgtagcagaaacattaaaaattgaagcatgttctcacagataataagcaccagtgatatttttacttttacaataatattatttgtggaattagtaataggaattttaggaaatggattcatagcactagtgaatatcatggactggaccaagagaagaagcatttcatcagcggatcagattctcactgctttggccattaccagatttctctatgtgtggtttatgatcatttgtatattgttattcatgctgtgcccacatttgcttacaagatcagaaatagtaacatcaattggtattatttggatagtgaataaccatttcagcgtttggcttgccacatgcctcggtgtcttttattttctgaagatagccaatttttctaactctttgtttctttacctaaagtggagggttaaaaaagtagttttaatgataatccaggtatcaatgattttcttgattttaaacctggtatctctaagcatgtatgatcagttctcaattgatgtttatgaaggaaatacattttataatttaggggattcaaccccatttcccacaatttccttattcatcaattcatcaaaagttttcgtaatcaccaactcatcccatattttcttacccatcaactccctgttcatgctcatacccttcacagtgtccctggtagcctttctcatgctcatcttctcactgtggaagcatcgcaaaaagatgcaggtcaatgccaagccacctagagatgccagcaccatggcccacattaaagccttgcaaacagggttgtccttcctgctgctgtatgcagtatacttactttttattgtcataggaatgttgagccttaggttgataggaggaaaattaatacttttatttgaccacatttctggaataggttttcctataagccactcatttgtgctgattctgggaaataacaagctgagacaagccagtctttcagtgttgcattgtctgaggtgccgatccaaagatatggacaccatgggtccataaaaaatttcagaggtcattgggaaacatcttgagatcttataggggaaaaagaaaacgtggggcttcaaagctggtaggagtaatatagagaaggataggaggagaatgaagagactaacattatatatgtgacctcagaggagaaggggagatctttaggaaataaagaggtacatacaggagtaagaggggttagagaggtaacataataataagaatggagttattctatgctaagaaaaatctatagtgcatctgattcatggctgccaagagactcctgaaacactcatcctattgctggtgatattgcttaattactagtggtgaaatttaaatccccatttcctgatgataccatgtattaacaaggaataggaaccttccgggttgggaactggacctgc

SEQ ID NO: 2 - protein, 333 aa, M. musculus
MFSQIISTSDIFTFTIILFVELVIGILGNGFIALVNIMDWTKRRSISSADQILTALAITRFLYVWFMIICILLFMLCPHLLTRSEIVTSIGIIWIVNNHFSVWLATCLGVFYFLKIANFSNSLFLYLKWRVKKVVLMIIQVSMIFLILNLVSLSMYDQFSIDVYEGNTFYNLGDSTPFPTISLFINSSKVFVITNSSHIFLPINSLFMLIPFTVSLVAFLMLIFSLWKHRKKMQVNAKPPRDASTMAHIKALQTGLSFLLLYAVYLLFIVIGMLSLRLIGGKLILLFDHISGIGFPISHSFVLILGNNKLRQASLSVLHCLRCRSKDMDTMGP

SEQ ID NO: 3 - DNA, 1634 bp, M. musculus
  misc_feature (1)...(1634): n = A,T,C or G
ctgagtaggatgacatataaataatatttatccattgatcaggactgatgttgtgtggcttaaaatgtgtatccagggccaaaagtgaaaataatgtgaataaatatatctctttttttgtagggttagcatcttgataattccagcatttttatttaacttcagaaaattagatgtgaagtactggataaagcagagttcatctctttgggaagaaaccaacccnagattttcatggaggaattatggaacaattcctggaaaattgatgtttcctaagagtaatgtgtgaatgggattttaaaggatgaatattttgagttttcagcgnccaatatgtagactttttaatgcatcagagacattatggattgaagcatgttttcacaggaaataaactgcagccatttgtttactttttcaatcaccttgtatgtggaaatagtaacgggaatcttaggacatggattcatagcattagtgaacatcatggactgggtcggaagaagaaggatctcttcagtggatcagattctcactgctttggcccttaccagattcatttatgtctgggctatgctgatttgcatattgttattcatgctgagcccacatttgcctaggagatcagaaatgctttcagcaatgggtattttctgggtagtcaacagccattttagcatctggcttactacatgcctcggtgtcttttattttctcaagatagccaatttttctaactctttttttctttatctaaagtggagagttaaaaaagtgattttaataataatcctggcatcactgattttcttgactttacacattttatctttagggatatatgatcagttctcaattgctgcttatgtaggaaatatgtcttatagtttgacagatttaacacaattttccagtactttcttattctccaactcatccaatgttttcttaatcaccaactcatcccatgttttcttacccatcaactccctggtcatgctcatacccttcacagtgtccctggtagcctttctcatgctcatcttctcactgtggaagcatcacaaaaagatgcaggtcaatgccagccaacctagaaatgtcagtactatggcccacattaaagccttgcaaactgtgttctccttcctgctgctgtatgccataaacttacttttccttatcataggaattttgaaccttggattgatggagaaaatagtgatcctgatatttgaccacatttctgcagcagtttttcctataagccactcatttgtactgattctgggaaacagtaagctgagacaagccagtctttctgtgttgccttgcctaaggtgccagtccaaagatatggacaccatgggtctctagtaaattccagagtacatttcgtaaaaatcccgaggacgatcagctcatagaaaaaagttaccttatgggggaaaataaaaagtggggcttcaatcctgggagcaataatacacaggagggcaggacagcatgaaggagactagcactatataagtggcctcatacaggatatgggaaaggaaagatttatgcaataaagagggagatacatattgcaggatgaggaggcactcacactacgtaaaatgactatc

SEQ ID NO: 4 - protein, 333 aa, M. musculus
MFSQEINCSHLFTFSITLYVEIVTGILGHGFIALVNIMDWVGRRRISSVDQILTALALTRFIYVWAMLICILLFMLSPHLPRRSEMLSAMGIFWVVNSHFSIWLTTCLGVFYFLKIANFSNSFFLYLKWRVKKVILIIILASLIFLTLHILSLGIYDQFSIAAYVGNMSYSLTDLTQFSSTFLFSNSSNVFLITNSSHVFLPINSLVMLIPFTVSLVAFLMLIFSLWKHHKKMQVNASQPRNVSTMAHIKALQTVFSFLLLYAINLLFLIIGILNLGLMEKIVILIFDHISAAVFPISHSFVLILGNSKLRQASLSVLPCLRCQSKDMDTMGL

SEQ ID NO: 5 - DNA, 1860 bp, M. musculus
attgagtttcattttttgtaccttggtaccctataccctcgcaaatgcaaagctggccccaaccaaatttcctttaaacttgatacaggcacaacatgcaaaacagtggaaaagttgtatcttgattagcatatctgcattgtgagtttgcatttatttcttaaatcatctgattaatatttaggatatggtaagggcaaacacatacttgctaagactcaccagagaattgcaggaaaaaaattacttaagaaaatatgtcaattaaatgccaaacaggaaatattcaacttgatatgttttcagagacttcaaaaggagcaggacaaagagaagaaaacatttaacagcacagtgaaaaactcatgggccacttggtcacccagggacaggcgacgctgttatatgccaagctttctatgaacatggaatctgtccttcacaactttgccactgtactaatatacgtggagtttatttttgggaatttgagcaatggattcatagtgttgtcaaacttcttggactgggtcattaaacaaaagctttccttaatagataaaattcttcttacattggcaatttcaagaatcactaacatctgggaaatatatgcttggtttaaaagtttatatgatccatcttcctttttaattggaatagaatttcaaattatttattttagctgggtcctttctagtcacttcagcctctggcttgccacaactctcagcgtcttttatttactcagaatagctaactgctcctggcagatctttctctatttgaaatggagacttaaacaactgattgtggggatgttgctgggaagcttggtgttcttgcttggaaatctgatgcaaagcatgcttgaagagagggtctatcaatatggaaggaacacaagtgtgaataccatgagcaatgaccttgcaatgtggaccgagctgatctttttcaacatggctatgttctctgtaataccatttacattggccttgatttcttttctcctgctaatcttttctttgtggaaacatctccagaagatgcagctcatttccagaagacacagagaccctagcaccaaggcccacatgaatgccttgagaattatggtgtccttcctcttgctctataccatgcatttcctgtctcttcttatatcatggattgctcaaaagcatcagagtgaactggctgatattattggtatgataactgaactcatgtatccttcagtccattcatgtatcctgattctaggaaattctaaattaaagcagacttctctttgtatgctgaggcatttgagatgtaggctgaaaggagagaatatcacaattgcatatagcaaccaaataactagcttttgtgtattctgtgttgcaaacaaatctatgaggtagttgttcaaggaatccttccttgacttattgtatcatggaagtcatatgggggagtctgaaagagctgtcttctgtaagcaaggtttgtatacactagtggggctgggacaccaacccaagcacaaaacctagctataacctatcctggctgcaggatatgctggaacaatggtggcttggaaattgtgggactggcaaagcaatagctagtctaacttgaggcccattccacagcaggaagctcatgcccacctctgcctggatggccaggaagcaaaatcttgatggccccaagacctatggtaaactgaacactactggaaaaaagaaagactcgtgtaatgatctatcaaatattttcctaatgatattctgataaactcatatattagtccctgtcctaatcatcatcactgggacttccttcccagcacctgatggggagcaaaaa

SEQ ID NO: 6 - protein, 347 aa, M. musculus
MGHLVTQGQATLLYAKLSMNMESVLHNFATVLIYVEFIFGNLSNGFIVLSNFLDWVIKQKLSLIDKILLTLAISRITNIWEIYAWFKSLYDPSSFLIGIEFQIIYFSWVLSSHFSLWLATTLSVFYLLRIANCSWQIFLYLKWRLKQLIVGMLLGSLVFLLGNLMQSMLEERVYQYGRNTSVNTMSNDLAMWTELIFFNMAMFSVIPFTLALISFLLLIFSLWKHLQKMQLISRRHRDPSTKAHMNALRIMVSFLLLYTMHFLSLLISWIAQKHQSELADIIGMITELMYPSVHSCILILGNSKLKQTSLCMLRHLRCRLKGENITIAYSNQITSFCVFCVANKSMR

SEQ ID NO: 7 - DNA, 1280 bp, M. musculus
atatttaaaaatccatttgaactgttttgtaaacattcttatttttataatacctgtaccatattcatccattagcacacagggatgctttcctacttgaaaatggccatgggatttctacaaaacatgatctttgctgaccataaatgaagaccacatgaatcagtgtgttcatgaaatcacagccagtgacacaacagctacctttcatttttcctcttttcaaaacttgctcagacatgatgagtttcttggtaagcattgcatccattgcaatgctggtgaaaattgttcttggaacctttgccaatgtcttcattgttctggtaaacttcactgactgcatcaagaaaagaaaattcctcttagctgatagaattctcactgttctggctatcttcaggtttgacttgctttggataatattaatgaattggagctcaagtgtgtttcatgtaggtttgtatttccaagtaagattttgtatttgtgttgtctggatagtaaccaaccattttaatacatggcttgcaaatatactcagcatactttatttgttgaagatagacaatttctcaaatcttatttttcttggcctgaaaggaaaaattaagtgtccttatattgtacttttgccatgttttgtgcttttatttcctaatcttataatggtaaccatatgtgagacaacacaagcaaatggacaccagggcaacttgactgggaagacaaaactgacttatttcacgaaccttatagctatgactttcactctaggcagtttagttcccttcaccacattcatgatctgtttccttctcttaatctgttctctgtgtaaacaccttaggacaatgaggctttatggaaaaggatcccagggccccagtgcttcaacccacattaaggttttgcaagttttgatctcatttctgttgttattctccatgtttattctgttgctaatcatatcagattacaattatacaaagtctctggaggaaccaatccacctgatttgccaggttattggaaccttgtatccttcaagacattcttatatcttgctatggggaaacaagaggatcaaacaggcctttgtgttggcaatggttcaggtgagagcaaggttctggctgaaagaaaagaaaccttgaaacacttcaatcaatttatgagatgcagtgtgactaatagcagggtctgtagcattgtattctttgtattgctttcaaaagtttactgtgtaaattgtttctaaagaaatttctagaaagcat

SEQ ID NO: 8 - protein, 305 aa, M. musculus
MMSFLVSIASIAMLVKIVLGTFANVFIVLVNFTDCIKKRKFLLADRILTVLAIFRFDLLWIILMNWSSSVFHVGLYFQVRFCICVVWIVTNHFNTWLANILSILYLLKIDNFSNLIFLGLKGKIKCPYIVLLPCFVLLFPNLIMVTICETTQANGHQGNLTGKTKLTYFTNLIAMTFTLGSLVPFTTFMICFLLLICSLCKHLRTMRLYGKGSQGPSASTHIKVLQVLISFLLLFSMFILLLIISDYNYTKSLEEPIHLICQVIGTLYPSRHSYILLWGNKRIKQAFVLAMVQVRARFWLKEKKP

SEQ ID NO: 9 - DNA, 672 bp, M. musculus
attggtgtcgtatggataataataatattacatgggaatatacaggtgcattatccacacacccacaccagaggaaacgaaacgaggaccgttgcctacttctggacacttaccaaccacttaagtgtctggtttgccacctgcctcagcattctctatttattcaagatagcaaacttcttccaccctcttttcctctggataaagaggagaattgacaagctaattctcagaactctactggcatgggtgaccatctgcctgcgttttaacctcccagtcactgaaaatctgagggatgatttcaaacgccgggggaacaccaaggagagaataacctctcctttgcgatgcaaagtaaataaagctggacatgcctctgtcaaggtaaatctcaacttggtcatgctgctccccttttctgtgtctctggtctcctttctcctcttggtcctctccctgtggagacacaccaggcagatacaactcagtgtaacagggtacaaagatcccagcacaacagctcatgtgaaagccatgaaagcagtaatttccttcctggccctgtttgttgtctactgcctagcctttctcatagccacctccagctactttatgccagagagtgaactacctgtaatatggggtgagctgatagctctaatctatcct

SEQ ID NO: 10 - protein, 224 aa, M. musculus
IGVVWIIIILHGNIQVHYPHTHTRGNETRTVAYFWTLTNHLSVWFATCLSILYLFKIANFFHPLFLWIKRRIDKLILRTLLAWVTICLRFNLPVTENLRDDFKRRGNTKERITSPLRCKVNKAGHASVKVNLNLVMLLPFSVSLVSFLLLVLSLWRHTRQIQLSVTGYKDPSTTAHVKAMKAVISFLALFVVYCLAFLIATSSYFMPESELPVIWGELIALIYP

SEQ ID NO: 11 - DNA, 675 bp, M. musculus
atttgtctacagtgtataatcctattagatggtattatattggtgcagtatccagacacttacaacaggggtaaagaaatgaggatcattgatttcttctggacgcttaccaaccatttaagtgtctggtttgccacctgcctcagcattttccatttcttcaagatagcaaacttcttccatcctcttttcctctggataaagtggagaattgacaagctaattctgaggactctactggcatgcttgattctctccctatgctttagcctcccagtcactgagaatttgactgatgatttcagacgctgtgtcaaaacaaaagaaagaataaactctactctgaggtgcaaattaaataaagctggatatgcttctgtcaaggtaaatctcaacttggtcatgctgttccccttttctgtgtcccttgtctcattccttctcttgattctctccctatggagacacaccaggcagatgcaactcaatgtaacagggtacaatgatcccagcacaacagctcatgtgaaagccacaaaagcagtaatttccttcctagttctgtttattgtctactgcctggcctttcttatagccacttccagctactttatgccagagagtgaattagctgtaatttggggtgagctgatagctctaatatatccctcaagc

SEQ ID NO: 12 - protein, 225 aa, M. musculus
ICLQCIILLDGIILVQYPDTYNRGKEMRIIDFFWTLTNHLSVWFATCLSIFHFFKIANFFHPLFLWIKWRIDKLILRTLLACLILSLCFSLPVTENLTDDFRRCVKTKERINSTLRCKLNKAGYASVKVNLNLVMLFPFSVSLVSFLLLILSLWRHTRQMQLNVTGYNDPSTTAHVKATKAVISFLVLFIVYCLAFLIATSSYFMPESELAVIWGELIALIYPSS

SEQ ID NO: 13 - DNA, 3 bp, M. musculus
  misc_feature (1)...(3): n = A,T,C or G
nnn

SEQ ID NO: 14 - protein, 3 aa, M. musculus
  VARIANT (1)...(3): Xaa = Any Amino Acid
XXX

SEQ ID NO: 15 - DNA, 912 bp, M. musculus
atgacctcccctttcccagctatttatcacatggtcatcatgacagcagagtttctcatcgggactacagtgaatggattccttatcattgtgaactgctatgacttgttcaagagccgaacgttcctgatcctgcagaccctcttgatgtgcacagggctgtccagactcggtctgcagataatgctcatgacccaaagcttcttctctgtgttctttccatactcttatgaggaaaatatttatagttcagatataatgttcgtctggatgttcttcagctcgattggcctctggtttgccacatgtctctctgtcttttactgcctcaagatttcaggcttcactccaccctggtttctttggctgaaattcagaatttcaaagctcatattttggctgcttctgggcagcttgctggcctctctgggcactgcaactgtgtgcatcgaggtaggtttccctttaattgaggatggctatgtcctgagaaacgcaggactaaatgatagtaatgccaagctagtgagaaataatgacttgctcctcatcaacctgatcctcctgcttcccctgtctgtgtttgtgatgtgcacctctatgttatttgtttctctttacaagcacatgcactggatgcaaagcgaatctcacaagctgtcaagtgccagaaccgaagctcatataaatgcattaaagacagtgacaacattcttttgtttctttgtttcttactttgctgccttcatggcaaatatgacatttagaattccatacagaagtcatcagttcttcgtggtgaaggaaatcatggcagcatatcccgccggccactctgtcataatcgtcttgagtaactctaagttcaaagacttattcaggagaatgatctgtctacagaaggaagagtga

SEQ ID NO: 16 - protein, 303 aa, M musculus
MTSPFPAIYHMVIMTAEFLIGTTVNGFLIIVNCYDLFKSRTFLILQTLLMCTGLSRLGLQIMLMTQSFFSVFFPYSYEENIYSSDIMFVWMFFSSIGLWFATCLSVFYCLKISGFTPPWFLWLKFRISKLIFWLLLGSLLASLGTATVCIEVGFPLIEDGYVLRNAGLNDSNAKLVRNNDLLLINLILLLPLSVFVMCTSMLFVSLYKHMHWMQSESHKLSSARTEAHINALKTVTTFFCFFVSYFAAFMANMTFRIPYRSHQFFVVKEIMAAYPAGHSVIIVLSNSKFKDLFRRMICLQKEE

SEQ ID NO: 17 - DNA, 1002 bp, M. musculus
atgagatttatgaacagaacaagcaaggatcagggtggcctaaattctaatatgtttggattcattgaaggggtgttcctggttctgactatcactgagtttattcttggaaatctggtgaatggtttcattgtgtcaatcaatagcagctattggttcaagagcaagaagatttctttgtctaacttcatcattaccagcttggccctcttcaggatctttctgttgtggattatctttattgatagtcttataatagtgttctcttaccagactcatgactcagggataatgatgcaactaattgatgttttctggacatttacaaaccacttcagtatttggcttatctcctgtctcagtgttttctactgcctgaaaatagccagtttctcccacccctcattcctctggctcaaatggagagcttctagagtggttgttgggatgctgtggggcgcactgctcttatcctgtgtcagtaccatgtctctgatgaatgaatttaagatctattctgccctcactagaagcaaagacacaccaaatatgactgaatacatcagactgaagcgacaggaatataatctgatgcatgttcttgggaatctgtggaagattccttccttaattgtttccctggttgcctaccttctgctgctcctctctctggggaagcacacacagcagatgcagcaatacagtattgactccagagatcagagtgctgaggcccacaaaagagccatgagaatcatctcttcctttctcctattcttcttattctactttctttcctttatgattttgtcatccagtcgtttcctaccagaaaccaggatcgccaggataattggagtagtgatttcaatgtcataccttgttggtgattcatttattctcatagtatgtaacaacaagctgaagcatacatttgtggccatgctcccatgtgagtgtggtcatctgaaacctggatctaagggaccctctgcttcataa

SEQ ID NO: 18 - protein, 333 aa, M. musculus
MRFMNRTSKDQGGLNSNMFGFIEGVFLVLTITEFILGNLVNGFIVSINSSYWFKSKKISLSNFIITSLALFRIFLLWIIFIDSLIIVFSYQTHDSGIMMQLIDVFWTFTNHFSIWLISCLSVFYCLKIASFSHPSFLWLKWRASRVVVGMLWGALLLSCVSTMSLMNEFKIYSALTRSKDTPNMTEYIRLKRQEYNLMHVLGNLWKIPSLIVSLVAYLLLLLSLGKHTQQMQQYSIDSRDQSAEAHKRAMRIISSFLLFFLFYFLSFMILSSSRFLPETRIARIIGVVISMSYLVGDSFILIVCNNKLKHTFVAMLPCECGHLKPGSKGPSAS

SEQ ID NO: 19 - DNA, 996 bp, M. musculus
atgctgagtctgactcctgtcttaactgtgtcctatgaagccaagatttcatttctgttcctttcagccatggagtttgcagtgggaatcctggccaacgccttcattgtcttggtaaatgtttgggatgtggtaaaaaagcagcccttgaacaactgtgacatcgcactgctgtgtctcagcatcactcggcttttcctgcagggccttctgcttctggatgctattcagctcgcctgcttccagcagatgaaagacccactgagccacaactaccaagccatcctcactctctggatgattgcaaaccaagtgagcctctggctggctgcctgcctcagtctcctctactgctccaagattgtccgcttctctcacacctttccactccatgtagcaagctgggtctccaggagatttcttcagatgcttctagttgttcttcttctctcctgcatctgcactgccctttgtttgtgggactttttttgcagatctcactccacggtcacatctctactgcacctgaacagcacagaattcagtttgcaaattgcaaaactcaatttcttttactcgtttatcttctgcaatgtgggctctgtccccccttctctagctttcctggtttcctcgggagtgctggttatctccctggggagtcacatgaggactatgaagtcccaaaccagcagctctggtgaccccagccttgaggcccacatcagagccatcatatttctgatctcctttttctgtttttacgtggtgtcattctgtgctgctttaatatcaatacccttactgatgctatggcacaataaggggggagtgatgatttgtatagggatgatggcagcttgtccttcgggacatgcagccatcctgatatcaggcaatgctaagctgaggagggccatagagaccatgctattctggtttcaaagcaggcaaaaggtgagaccagtccacaaggttcctcccaggacactctga

SEQ ID NO: 20 - protein, 331 aa, M. musculus
MLSLTPVLTVSYEAKISFLFLSAMEFAVGILANAFIVLVNVWDVVKKQPLNNCDIALLCLSITRLFLQGLLLLDAIQLACFQQMKDPLSHNYQAILTLWMIANQVSLWLAACLSLLYCSKIVRFSHTFPLHVASWVSRRFLQMLLVVLLLSCICTALCLWDFFCRSHSTVTSLLHLNSTEFSLQIAKLNFFYSFIFCNVGSVPPSLAFLVSSGVLVISLGSHMRTMKSQTSSSGDPSLEAHIRAIIFLISFFCFYVVSFCAALISIPLLMLWHNKGGVMICIGMMAACPSGHAAILISGNAKLRRAIETMLFWFQSRQKVRPVHKVPPRTL

SEQ ID NO: 21 - DNA, 960 bp, M. musculus
atggctcaacccagcaactactggaaacaagatgtgctaccattgtctattttgatgttaacacttgtggccactgagtgcaccataggtatcattgcaagtgggattgtcatggctgtgaatgcagtctcatgggttcagaaaaaggcaatttccataactactaggattctgcttcttctgagtgtatccagaataggcctccaaagcatcatgttgatagaaattacctcctccatattcaacgttgctttttacaacagtgttttatatagagtctcaaatgtaagttttgtattcttaaattattgtagtctctggtttgctgctttgcttagtttcttccactttgtgaagattgccaatttttcttaccccctgttcttcaaactaaagtggagaatttctgaattaatgccctggcttctgtggctctcagtgtttatttccttcagctccagcatgttcttcagcaagcacaagttcactgtgaacaacaacaattctctaagtaacaacatctgcaacttcacaatgaaactttacgttgttgagaccaatgtggtcaatgtgtcttttttattcatttcgggaatactccctcctttgacaatgttcgtcgcaacagctactcttctgattttttctctcaggagacacaccctgaacatgagaaacagtgccactggctccagaaacccctgcatagaggctcatatgcaggccatcaaagaaactagctgttttctctttctctacattttaaatgcagctgctctgcttctgtccacatccaacatagtcgatgctagtctcttctggagtattgtgatcagaattgttctgcctgtctacccagctggccattcagttttactaattcagaacaaccctggattaagaagaacatggaagcatcttcagtctcaaattcatttgtacttacaaaatagattctga

SEQ ID NO: 22 - protein, 319 aa, M. musculus
MAQPSNYWKQDVLPLSILMLTLVATECTIGIIASGIVMAVNAVSWVQKKAISITTRILLLLSVSRIGLQSIMLIEITSSIFNVAFYNSVLYRVSNVSFVFLNYCSLWFAALLSFFHFVKIANFSYPLFFKLKWRISELMPWLLWLSVFISFSSSMFFSKHKFTVNNNNSLSNNICNFTMKLYVVETNVVNVSFLFISGILPPLTMFVATATLLIFSLRRHTLNMRNSATGSRNPCIEAHMQAIKETSCFLFLYILNAAALLLSTSNIVDASLFWSIVIRIVLPVYPAGHSVLLIQNNPGLRRTWKHLQSQIHLYLQNRF

SEQ ID NO: 23 - DNA, 960 bp, M. musculus
atggcaataattaccacaaattctgactattttgctcacaggtatgaagtcataatccctttcgtggtctctacaatatgctctattgttggcatcattggcaatggcttcatcacagtcatctatgggactgaatgggtcaggagcaaaagactccccactggtgagaaccttatgttgatgctgagtttttccaggctgttgctacagatatggatgatggtagagattacttatagtctacttttcccgatcatttataaccataatgccatgtataaactattcaaagccatctctgtgtttctaaactactgtaacctctggtttgctgcttggctcaatgtcttctattgtcttaaaattgtgaacttagctcaccctctgttccttctgatgaagcagaaaatcatagggctgatgcctcggctcctgagtctgtcagtgttggtttccttcagcttaagttccttcttctctaaagacatcttaaatgtgtatgtgaacacttctgttcccatcccttcttccaactccacaaagatgaagtacatctttatgatcaatgtactcagcctagctttcttgtattatatggggatcttccttcctttgttcatgttcatcatggcagccactctgctgatcacctcactcaagaggcacaccctgcacatggaaaacagcaccacaggctctagggactccagcatggaggctcacgtgggtgccatcaaatcgaccagccactctctcattctctacattattaatgcactggctttatttatttccatgtcaaacatccttggtgcttacagtgtctggaatagtttgtgcaacattatcatgactgcctatccagccggccagtcagtgcatctgatcttgagaaatccagggctgagaagagcctggaggcggtttcagcaccatgttcatctttaccttaaaaggtag

SEQ ID NO: 24 - protein, 319 aa, M. musculus
MAIITTNSDYFAHRYEVIIPFVVSTICSIVGIIGNGFITVIYGTEWVRSKRLPTGENLMLMLSFSRLLLQIWMMVEITYSLLFPIIYNHNAMYKLFKAISVFLNYCNLWFAAWLNVFYCLKIVNLAHPLFLLMKQKIIGLMPRLLSLSVLVSFSLSSFFSKDILNVYVNTSVPIPSSNSTKMKYIFMINVLSLAFLYYMGIFLPLFMFIMAATLLITSLKRHTLHMENSTTGSRDSSMEAHVGAIKSTSHSLILYIINALALFISMSNILGAYSVWNSLCNIIMTAYPAGQSVHLILRNPGLRRAWRRFQHHVHLYLKR

SEQ ID NO: 25 - DNA, 882 bp, M. musculus
atgccctccacacccacattgatcttcattatcatcttttacctggtgtcattggcctctatgttgcagaatggcttcatgatgattgtgctgggcagagagtggatgaggaaccggacactaccggcagctgacatgattgtggcctctcttgcttcctcccggttctgcttgcatgggatcgccatcctggccaacctcttggcctcctttgatttttgttaccaagcgaaccttattggcatcctctgggatttcactaacactctcattttttggcttactgcctggcttgccatcttctactgtgtgaagatctcctctttctcccaccctgtcctcttttggctcaagtggaggatttcccagttagttcccaggctgctggttgtatctctcatcataggtggcctgtcagctgtcatttcagccaccgggaacttcatggccaatcagatgaccatctcccagggtttccatggaaactgcacttttggtcacatgtcactggacttctatcggtactattacctgtatcactcagtgctcatgtggttcactcctttcttcctgtttctagtgtccgttatcgtgctcatgttctcactgtaccagcatgtggagaagatgaggggccacaggcctgggccttgggatctccatactcaggcacataccatggctctgaaatcccttaccttcttcttcatcttttatatcttttttttcttggccctggtaatttctagtacaaaaaggaaaagcatgcagagttactattgggccagagaggctatcatctacacaggcatctttttgaactccatcatcctgctgtttagcaaccccaaactgagaaaggccctgaagatgaggttttag

SEQ ID NO: 26 - protein, 293 aa, M. musculus
MPSTPTLIFIIIFYLVSLASMLQNGFMMIVLGREWMRNRTLPAADMIVASLASSRFCLHGIAILANLLASFDFCYQANLIGILWDFTNTLIFWLTAWLAIFYCVKISSFSHPVLFWLKWRISQLVPRLLVVSLIIGGLSAVISATGNFMANQMTISQGFHGNCTFGHMSLDFYRYYYLYHSVLMWFTPFFLFLVSVIVLMFSLYQHVEKMRGHRPGPWDLHTQAHTMALKSLTFFFIFYIFFFLALVISSTKRKSMQSYYWAREAIIYTGIFLNSIILLFSNPKLRKALKMRF

SEQ ID NO: 27 - DNA, 939 bp, M. musculus
atgagcacaggccatacagttcttggatgtcagactactgataagacagtcgtcaccttatttatcattttagtccttttgtgcctggtggcagtggtaggcaatggatttatcattatagcactgggcatgaaatggttgctccggagaacactgtcagctcataataagttactgatcagtctagcagcctctcgattctgtctccaatgtgtggtgataggtaagaatatttatgttttcctgaatccaacaagcttcccatacaaccctgtaatacagctcctaaatttaatgtgggacttcttgactgctgcaaccatctggctctgttctttgctaggtttcttctattgtgtgaaaattgcaaccttaacccatcctgtctttgtctggctaaagtacaggttgcctgggtgggtaccatggatgctgctcagtgctgtggggatgtcgagcttaactagtatcctatgtttcataggcaattatatgatatatcagaaccatgcaaagagtggccatcaaccttggaatgtcactgggaatagcttaagacactcacttgagaaattctacttcttttctataaagataatcatgtggacaattcccactgttgtcttcagcatcttcatgagtttgctcctcgtatctttggtaagacacatgaagaagactttcttggccctttcagaacttcgggatgtctgggcacaggcccatttcaaggctcttcttcctctgctctccttcatcgtccttttcatctcctgttttctgacgctggtactcagttctgccagcaacacaccatatcaggaattcaggtactggatgtggcaggtggtgattcatctgtgcacagtgatacatcccattgttatactcttcagcaaccctgttttgagagtggtgataaagaggggctgctgctga

SEQ ID NO: 28 - protein, 312 aa, M. musculus
MSTGHTVLGCQTTDKTVVTLFIILVLLCLVAVVGNGFIIIALGMKWLLRRTLSAHNKLLISLAASRFCLQCVVIGKNIYVFLNPTSFPYNPVIQLLNLMWDFLTAATIWLCSLLGFFYCVKIATLTHPVFVWLKYRLPGWVPWMLLSAVGMSSLTSILCFIGNYMIYQNHAKSGHQPWNVTGNSLRHSLEKFYFFSIKIIMWTIPTVVFSIFMSLLLVSLVRHMKKTFLALSELRDVWAQAHFKALLPLLSFIVLFISCFLTLVLSSASNTPYQEFRYWMWQVVIHLCTVIHPIVILFSNPVLRVVIKRGCC

SEQ ID NO: 29 - DNA, 897 bp, M. musculus
atgtctttctcacattcattcatcttcatagtcatcttttgtatgcagtctctagctgctttgctgcaaaatggctttatggccaccgtgctgggcagggaatgggtacgaagccagggcctccctgcaggtgacatgattatggcttgcttagctgcctccaggttctgtctgcatggaatagccgtcctaaacaacttcctggcctctgctatgttttggaccataaagaattatttttctatcctctgggacttcaccaacactgtcaatttctggtttaccacctgtcttgctatcttctactgtgtaaagatctcttcgttttcccaccccatcttcttctggataaaatggagaatttctcggtcagtgcccaggttactgctgggatccctgatcattggtggactgtcagccatctcctcagccactggaaacacaattgcccttcagatggcggcctgtgaaaactacacaatttattataaaacgatggcattttatctgtattattttcgctgtcatgcgatgctgatgtgggtcattccattcttcctgtttctgctgtccatcatcttgctcatgttctcactgtatcggcatctggaacagatgaggtaccacagacccaggactcatgattatagcacccaggctcacattatggctctgaagtcccttgccttcttcctcatcttctatacatcatatgccctgctccttatggtatctgttgcacatgtcataaatgtccacggttcctggcactgggcctgggaagtggtaacctacatgggcatctcactgcattccaccattctgatactaagcaacaccaagatgagaaaggccctcatgataaagttcccagacctttgtattcccagatcataa

SEQ ID NO: 30 - protein, 298 aa, M. musculus
MSFSHSFIFIVIFCMQSLAALLQNGFMATVLGREWVRSQGLPAGDMIMACLAASRFCLHGIAVLNNFLASAMFWTIKNYFSILWDFTNTVNFWFTTCLAIFYCVKISSFSHPIFFWIKWRISRSVPRLLLGSLIIGGLSAISSATGNTIALQMAACENYTIYYKTMAFYLYYFRCHAMLMWVIPFFLFLLSIILLMFSLYRHLEQMRYHRPRTHDYSTQAHIMALKSLAFFLIFYTSYALLLMVSVAHVINVHGSWHWAWEVVTYMGISLHSTILILSNTKMRKALMIKFPDLCIPRS

SEQ ID NO: 31 - DNA, 939 bp, r. rattus
atgacatatgaaactgatactaccttaatgtttgtagctgtttgtgaggccttagtaggaatcttaggaaatgcattcattgcattggtaaacttcatgggctggatgaagaataggaagatcactgctattgatttaatcctctcaagtctggctatgtccaggatttgtctacagtgtataattctattagattgtattatattggtgcagtatccagacacttacaacaggggtaaagaaatgaggatcattgatttcttctggacgcttaccaaccatttaagtgtctggtttgccacctgcctcagcattttctatttcttcaagatagcaaacttcttccatcctcttttcctctggataaagtggagaattgacaagctaattctgaggactctactggcatgcttgattctctccctatgctttagcctcccagtcactgagaatttggctgatgatttcagacgctgtgtcaagacaaaagaaagaataaactctactctgaggtgcaaattaaataaagctggatatgcttctgtcaaggtaaatctcaacttggtcatgctgttccccttttctgtgtcccttgtctcattccttctcttgattctctccctatggagacacaccaggcagatgcaactcaatgtaacagggtacaatgatcccagcacaacagctcatgtgaaagccacaaaagcagtaatttccttcctagttctgtttattgtctactgcctggcctttcttatagccacttccagctactttatgccagagagtgaattagctgtaatttggggtgagctgatagctctaatatatccctcaagccattcatttatcctgatccttgggaacagtaaactaaaacaggcatctgtaagggtgctttgtagagtaaagactatgttaaagggaagaaaatattag

SEQ ID NO: 32 - protein, 312 aa, R. rattus
MTYETDTTLMFVAVCEALVGILGNAFIALVNFMGWMKNRKITAIDLILSSLAMSRICLQCIILLDCIILVQYPDTYNRGKEMRIIDFFWTLTNHLSVWFATCLSIFYFFKIANFFHPLFLWIKWRIDKLILRTLLACLILSLCFSLPVTENLADDFRRCVKTKERINSTLRCKLNKAGYASVKVNLNLVMLFPFSVSLVSFLLLILSLWRHTRQMQLNVTGYNDPSTTAHVKATKAVISFLVLFIVYCLAFLIATSSYFMPESELAVIWGELIALIYPSSHSFILILGNSKLKQASVRVLCRVKTMLKGRKY

SEQ ID NO: 33 - DNA, 945 bp, r. rattus
atgactgcaacagtagttaatgtggagttcatacttggaaatttggggaatggattcatcgctgtggcaaacataatggattgggtcaagagaaggaagctctctgcagtggatcagctcctcactgtgctggccatctccagaatcactctgttgtggtcattgtacatactgaaatcaacattttcaatggtgccaaactttgaagtagctataccgtcaccaagactaactaatcttgtctggataatttctaaccattttaatatatggctagccaccattctcagcatcttttattttctcaagataggaaatttttctaactctatattctattacctaagatggagatttaaaaaggtggttttggtggcactactggtgtctctggtcctcttgtttatagatatttttgtcacaaacatacacatcaatatctggaaagatgaattcaaagcaaacgtatcttacagttacaaattaaagatctttttacaggtttccaggcttctggtggtaactaatactatgttcgcatgtgtacctttcgttgtgtccatgataatgttttttctactcatcttctccctgtggaaaaatctgaagatgatgaagcacattgcccaaagctcccaaaatgccagcactacagcccacatcaatgccttgaaaactgttgttgccttcctcctgctgtatatcatttttattttatccctttttgcacatgtttggagctatgactttgaagaaaagaaatattttattttcttttgccttgttggtatgtttgcattaccatcactccattcatacatcttgattctgggaaacagtaagttgaggcagatctctcttttggtactgtcactgctaaagtgcaagatccaaggatgtgaatccctgggcccctggcacactaggggggatacttttacataa

SEQ ID NO: 34 - protein, 314 aa, R. rattus
MTATVVNVEFILGNLGNGFIAVANIMDWVKRRKLSAVDQLLTVLAISRITLLWSLYILKSTFSMVPNFEVAIPSPRLTNLVWIISNHFNIWLATILSIFYFLKIGNFSNSIFYYLRWRFKKVVLVALLVSLVLLFIDIFVTNIHINIWKDEFKANVSYSYKLKIFLQVSRLLVVTNTMFACVPFVVSMIMFFLLIFSLWKNLKMMKHIAQSSQNASTTAHINALKTVVAFLLLYIIFILSLFAHVWSYDFEEKKYFIFFCLVGMFALPSLHSYILILGNSKLRQISLLVLSLLKCKIQGCESLGPWHTRGDTFT

SEQ ID NO: 35 - DNA, 885 bp, r. rattus
atgccctccacacccacattgatcttcattgtcatcttttttctggtatcagtggcctctatgttgcagaatggcttcatgatcattgtgctgggcagagagtggatgaggaaccgggcactgccggcagttgacatgattgtggcttctcttgcttcctcccggttctgcctacatgggatagccatcctcaacaatttcttggcctcctttgatttttgttaccaagcaaactttgttggcatcctctgggacttcattaatactctcattttgtggcttactgcctggcttgccatcttctactgtgtgaagatctcctctttctcccaccctgtcctcttttggctcaagtggaggatttcccagttagttcccaggctgctgctggtatctctcatcatgggtggcctgtcagccatcatatcagctaccgggaacatcattgccaatcagatgatcatctcccaaggtttccatggaaactgcacttttggtcacatgtcactggacttctatcggtattattacctgtctcacgcagtgctcatgtggttcactcctttcttcctgtttctagtgtccattatcttcctcatgttctcactgtaccggcatgtggagaagatgaggggccataggcctgggccttgggatccccgtacacaggcacacaccatggctctgaaatcccttactgtcttcatcaccttctatatattattttttctggccctgataatttctagtacaaaaagtaaaactatgcacagttactggtattgggtccgagaaattatcatctacactggcatctttttgaactccatcatcttggtgcttagcaaccccaagctgagaaaggccctgaagatgagattttag

SEQ ID NO: 36 - protein, 294 aa, R. rattus
MPSTPTLIFIVIFFLVSVASMLQNGFMIIVLGREWMRNRALPAVDMIVASLASSRFCLHGIAILNNFLASFDFCYQANFVGILWDFINTLILWLTAWLAIFYCVKISSFSHPVLFWLKWRISQLVPRLLLVSLIMGGLSAIISATGNIIANQMIISQGFHGNCTFGHMSLDFYRYYYLSHAVLMWFTPFFLFLVSIIFLMFSLYRHVEKMRGHRPGPWDPRTQAHTMALKSLTVFITFYILFFLALIISSTKSKTMHSYWYWVREIIIYTGIFLNSIILVLSNPKLRKALKMRF

SEQ ID NO: 37 - DNA, 915 bp, r. rattus
atgctctgggaactgtatgcatttgtgtttgccgcctcagttgtttttaattttgtaggaatagttgcaaatttatttattatagtgataatttctaagacttgggtcaaaagtcacaaaatctcctcttcagataagatcctgttcagcttggccatcactagattcctgaccctggggttgtttctactgaacactgtctacattgctacaaacactggaaggtcagtctacttttccacgttttttctcttgtgttggaagtttctggactccaacagtctctggctagtgacctttctgaactgcttgtattgcgtgaagatcactcatttccaacatccagtgtttcttctgttgaaacggactgtctctatgaagaccaccagcctgctgctggcctgccttctgatttctgccttcaccactctcctatattttgtgctcacacagatatcacgttttcctgaacacataattgggagaaatgacacattatttgacgtcagtgatggcatcttgacgttagcggcttctttgatcctgagctcacttttacagtttctgctcaatgtgacctttgcttctttgctaatacattccctgagaagacatgtacagaagatgcagagaaacaggagcagcttttggaatccccagacggaggctcacgtgggcgccatgaggctgatgatctgtttcctcgtgctctatattccatattcaatcgctgccttgctctatttcccttcctatatgaggaagaatctgagagcccaggctgcttgcatgatcattactgctgcttaccctccaggacattctatcctccttattatcacacatcacaaactgaaagctaaagcaaagaagatttgctgtttctacaaattgcgggatttcgttagtaactga

SEQ ID NO: 38 - protein, 304 aa, R. rattus
MLWELYAFVFAASVVFNFVGIVANLFIIVIISKTWVKSHKISSSDKILFSLAITRFLTLGLFLLNTVYIATNTGRSVYFSTFFLLCWKFLDSNSLWLVTFLNCLYCVKITHFQHPVFLLLKRTVSMKTTSLLLACLLISAFTTLLYFVLTQISRFPEHIIGRNDTLFDVSDGILTLAASLILSSLLQFLLNVTFASLLIHSLRRHVQKMQRNRSSFWNPQTEAHVGAMRLMICFLVLYIPYSIAALLYFPSYMRKNLRAQAACMIITAAYPPGHSILLIITHHKLKAKAKKICCFYKLRDFVSN

SEQ ID NO: 39 - DNA, 898 bp, r. rattus
atgtccagcctacaggagattttggttgtgatcttttctgttatagaattcataatgggaactttgggaaatggatttattgtactgataaacagtacttcctggttcaagagtcggagaatctctgtaattgattttattctcacttgctcggccatctcgagaatgtgtgttttgtggacaacagttgcaggtgtctctttctacaaggcattattttactttaaaagtttgcaagtattttttgacattatctggacaggatccaactatttatgtacagcctgtacaacctgcatcagtgtcttctacttgttcaagatagccaacttttctaatccaattttcatctgggttaaacagagaattcataaggtgcttctgtctattgttctaggaacaatcatctatttcttcttatttctcatttttatgaaaataataactaattattttatatacgactggacaaaattggaacaaaacaaaacattcacttttttagatactctaactggtttcttagtctacaatgtgattctcatattttttttttgcagtgtctctgacatcgtttcttcttttaatcttctctttacggagccacctcaggaggatgaaactacagggcatacataccaaggacacaagcacagaagcacacataagagctatgaaaattataatttcattcctcttgttcttcatcatatattatatcagcaacactatgcttactttttcacattccattcttgacaatgtggttccaaaaactttctcttatatcctaacatttatgtatttatctattcatccctttctcctggttttatggaacagcaaattgaaatgggcattccagtgtgtattgagaaagctagtgtgtcatagttga

SEQ ID NO: 40 - protein, 296 aa, R. rattus
MSSLQEILVVIFSVIEFIMGTLGNGFIVLINSTSWFKSRRISVIDFILTCSAISRMCVLWTTVAGVSFYKALFYFKSLQVFFDIIWTGSNYLCTACTTCISVFYLFKIANFSNPIFIWVKQRIHKVLLSIVLGTIIYFFLFLIFMKIITNYFIYDWTKLEQNKTFTFLDTLTGFLVYNVILIFFFCSVSDIVSSFNLLFTEPPQEDETTGHTYQGHKHRSTHKSYENYNFIPLVLHHILYQQHYAYFFTFHSQCGSKNFLLYPNIYVFIYSSLSPGFMEQQIEMGIPVCIEKASVS

SEQ ID NO: 41 - DNA, 733 bp, r. rattus
atggatttgacagaatggatcgtcactatcataatgatgatagaatttctcttaggaaactgtgctaatttcttcataatggtagtgaacgccattgactgtatgaagagaagaaagatctcctcagccgatcgaattataactgctcttgccatctccagaattggtttgttatgggcaatgttaatgaactggcattcacgtgtgtatactacagatacgtacagttttcaagtgacagcttttagtggaattatctgggcgataactaatcattttaccacttggcttgggaccatactcagcatgttttatttattcaagatagccaacttttccaattgtctatttcttcatctgaaaagaaaacttgacagtgttcttcttgtgatatttttggtgtcttctttgcttgtgtttgcataccttggggtagtgaacatcaagaagattgcttggttgagtgttcatgaaggaaatgtgacggtaaagagcaaactgatgaatatagcaagcattagagatacgcttctcttcagcctgataaacatcgcaccatttggtatatcactgacctgtgttctgctcttaatctactccctaggcaaacatctcaagaatatgaaattctatggcaaaggatgtcaagatcagagtaccatggtccacataagggccttgcaaactgtggtttcctttctcttgttatatgctacatactcttcctgtgtaa

SEQ ID NO: 42 - protein, 244 aa, R. rattus
MDLTEWIVTIIMMIEFLLGNCANFFIMVVNAIDCMKRRKISSADRIITALAISRIGLLWAMLMNWHSRVYTTDTYSFQVTAFSGIIWAITNHFTTWLGTILSMFYLFKIANFSNCLFLHLKRKLDSVLLVIFLVSSLLVFAYLGVVNIKKIAWLSVHEGNVTVKSKLMNIASIRDTLLFSLINIAPFGISLTCVLLLIYSLGKHLKNMKFYGKGCQDQSTMVHIRALQTVVSFLLLYATYSSCV

SEQ ID NO: 43 - DNA, 960 bp, r. rattus
atggcaataataaccacagattccgactactatactcacaggtatgaagtgataatccctttcgtggtctcgaccatagattgtatcgtcggcatcattggcaatggcttcatcacagtcatatatgggactgaattggtcaggagcaaaagactccccactggtgagcaccttatgttgatgttgagtttttccaggctcttgctacagatctggataatggtagagattacctatcaactatttttccccatgatttataaccataatgccatgtataaactattcaaaaccatctctgtgtttctgaactactgtaacctctggtttgccgcgtggctcaatgtcttctattgtcttaaaattgtgaactttgctcaccctctgtttcttatgatgaagcagaaaatcgtagtgttgatgcctcggctcatgagtctgtcagtgttggtttccatcagcttaagctccttcttctctaaagacatcttcaatgtgtatatgaatacttcagttcccatccctttctccaactccacaaagatgaagtacttctttaagaccaatgtactcaacctggctttcttatattatatggggatcttcattcctttgttcatgttcatcatggcagccattctgctcatcacctcactcaagaggcacaccctgaacatggaaagcagcaccacaggctctagggactccagcatggaggctcacttgggtgccatcaaatcgaccagctactctctcattctctacattatcaatgcactggctctatttatttccatgtcaaatatctttggtgcctatagtacctggaatagtgtgtgcagctttatcctgaccgcctatccagctggacagtcagtgcatctgatcttgagaaacccagggctgagaagagcctggaggcggtttcagcaccacgttcgactttaccttaaaagatag

SEQ ID NO: 44 - protein, 319 aa, R. rattus
MAIITTDSDYYTHRYEVIIPFVVSTIDCIVGIIGNGFITVIYGTELVRSKRLPTGEHLMLMLSFSRLLLQIWIMVEITYQLFFPMIYNHNAMYKLFKTISVFLNYCNLWFAAWLNVFYCLKIVNFAHPLFLMMKQKIVVLMPRLMSLSVLVSISLSSFFSKDIFNVYMNTSVPIPFSNSTKMKYFFKTNVLNLAFLYYMGIFIPLFMFIMAAILLITSLKRHTLNMESSTTGSRDSSMEAHLGAIKSTSYSLILYIINALALFISMSNIFGAYSTWNSVCSFILTAYPAGQSVHLILRNPGLRRAWRRFQHHVRLYLKR

SEQ ID NO: 45 - DNA, 930 bp, r. rattus
atggtggccgttctacagagcacatttgcaataattttcagtatggagttcatagtgggaaccttaggaaatggattcattattctgatgacatgcatagactgggtccgaagaagaaaaatctctttagtggatcaaatcctcactgctctggcaattaccagaatcactctaattttgttggtattcatagattggtgggtatctgttcttttcccagcattacatgaaactggtaagatattaagaatgtattttatctcctggactgtgatcaatcattgtaatctttggttgacagcaagcctgagcatcatttattttctcaagatagccagcttttctagcattatttttctttatctaaagtttagagttaaaaatgtggtttttgtgaccttgttagtgtctctatttttcttgttcataaatactgctattgtaaatgtatattttgatgtttgttttgatggtgttcaaagaaatgtgtctcaagtttccagattgtataaccacgaacaaatttgcaaatttctttcttttactaaccctatgtttgcattcataccctttgttacgtccatggcaacgttctttctgctcatcttctccctgtggagacatctgaaaaacatgaagcacaacgcagaaggatgcagagacgtcagcaccatagtacacatcagagccttgcaaaccatcattgtgtctgtagtgttatacagtacttttttcctgtcattttttgtaaaagtttggagttctgggtcaccggagagatacctgatctttctgtttgtctgggctctgggaaatgctgttcttcctgctcacacgtttgtcctgatttggggaaactgtagattgaggtgggcctctctctccctgatgttgtggctcaggtacaggttcaaaaatatagacgtatag

SEQ ID NO: 46 - protein, 309 aa, R. rattus
MVAVLQSTFAIIFSMEFIVGTLGNGFIILMTCIDWVRRRKISLVDQILTALAITRITLILLVFIDWWVSVLFPALHETGKILRMYFISWTVINHCNLWLTASLSIIYFLKIASFSSIIFLYLKFRVKNVVFVTLLVSLFFLFINTAIVNVYFDVCFDGVQRNVSQVSRLYNHEQICKFLSFTNPMFAFIPFVTSMATFFLLIFSLWRHLKNMKHNAEGCRDVSTIVHIRALQTIIVSVVLYSTFFLSFFVKVWSSGSPERYLIFLFVWALGNAVLPAHTFVLIWGNCRLRWASLSLMLWLRYRFKNIDV

SEQ ID NO: 47 - DNA, 984 bp, r. rattus
atggaacctgtcatttacagctttgccactctactaatacatgtggagttcatttttgggaatctgagcaatggatttatagtgttgtcaaacttctgggactgggtcattaaacgaaaactttccacaattgataaaattcttcttacattggcaatttcaagaatcactctcatctgggaaatatatacttggtttacaagtgtatatggtccatcttcatttgcaattggaatgaaattacaaattctttattttacctggatcctttctagtcacttcagcctctggtttgccacagctctcagcatcttttacttactcagaatagctaactgctcctggaagatcttcctgtatttgaaatggagacttaaacaagtgattgtggggatgttgttggcaagcttggtgttcttgcctggaatcctgacgcaaaggactcttgaagagaggccctatcgatatggaggaaacacaagtgaggattccatggaaactgactttgcaaggtttacagagctgattcttttcaacttgactatattctctgtaataccattttcattggcctcgatttcttttctcctgctaatcttctccttgtggaaacatctccggaagatgcagctcagttccagaggacatggagaccctagcaccaaggcccacacaaatgctttgagaattatggtctccttcctcttgctctattctatatatttcctgtctcttcttttatcatggattgctcagaagcatcacagtaaactggttgacattattggtattattactggactcatgtatccttctgcccactcatttattctgattctaggaaattctaaattaatgcagacttctctttggatactgagtcatttgagatgtagactgaaaggagagaatattttaaatccatctggcaaccaagtaactagctgttatatattctgtattgcgaataaatctgtgagttag

SEQ ID NO: 48 - protein, 327 aa, R. rattus
MEPVIYSFATLLIHVEFIFGNLSNGFIVLSNFWDWVIKRKLSTIDKILLTLAISRITLIWEIYTWFTSVYGPSSFAIGMKLQILYFTWILSSHFSLWFATALSIFYLLRIANCSWKIFLYLKWRLKQVIVGMLLASLVFLPGILTQRTLEERPYRYGGNTSEDSMETDFARFTELILFNLTIFSVIPFSLASISFLLLIFSLWKHLRKMQLSSRGHGDPSTKAHTNALRIMVSFLLLYSIYFLSLLLSWIAQKHHSKLVDIIGIITGLMYPSAHSFILILGNSKLMQTSLWILSHLRCRLKGENILNPSGNQVTSCYIFCIANKSVS

SEQ ID NO: 49 - DNA, 1011 bp, r. rattus
atgaaatcacaaccagtgacacaagagctacatttcatttttcctcttttcaaaactatttcttcagacataatgagtttcttggtaagcattgcaggcattgcgatgctggcacaaattgttcttggcacctttgccaatgtcttcattgttctggtgacctgcactgactgcatcaggagaagaaaattgttcctggctgatggaattctcacttccctggccttctgcaggattggcatgctctgggtaatattaataagttggtgctcaattgtgtttcaccaagctttgtctttacaagtaagatttagcatttgcgttggctgggcagtaaccaaccattttaatatgtggcttgccactatacttagcatactttatttgttgaagataggtaatttctctaatcttatttttcttggcctaaagagaaaaatcaagagtgtctttatagttgtacttttggcgagcttggtgcttttatttcctaatcttataacggtaaccgtatgtgagacagtacaagcgaatggataccgaggcaacttgactgggaagaccaaacggacttatttcatgaaccttacagctatgatatcttttactctagacaacatcatttccttcaccatatccatggtctgttttcttctgttaatctattccctgtgtaaacaccttaggacaatgaggctttatggaaaaggaccccacaacccgagtgcgtcagcccacattaaggctctgcaagctgtgatctcctttctgttgttattttccatgtttattctgtctctaatcatatcaggttacaattatatgaagcctctaaatgaaccagtccacctgatttgccagcttattgggactttgtatccttcaagccattcttacgttttgctatggggaaataggaggatcaaactggcctttgtgttggctatggtacaggtgagggcaaggctctggctaaaagagaagaaccttgaagtccttcaaccaatttaa

SEQ ID NO: 50 - protein, 336 aa, R. rattus
MKSQPVTQELHFIFPLFKTISSDIMSFLVSIAGIAMLAQIVLGTFANVFIVLVTCTDCIRRRKLFLADGILTSLAFCRIGMLWVILISWCSIVFHQALSLQVRFSICVGWAVTNHFNMWLATILSILYLLKIGNFSNLIFLGLKRKIKSVFIVVLLASLVLLFPNLITVTVCETVQANGYRGNLTGKTKRTYFMNLTAMISFTLDNIISFTISMVCFLLLIYSLCKHLRTMRLYGKGPHNPSASAHIKALQAVISFLLLFSMFILSLIISGYNYMKPLNEPVHLICQLIGTLYPSSHSYVLLWGNRRIKLAFVLAMVQVRARLWLKEKNLEVLQPI

SEQ ID NO: 51 - DNA, 770 bp, r. rattus
cttttgtgtggtcactactcacagttttagtgatatctgagcttcactcatcattgttgataacaagaaaaatgttgaggataatcaataatttctggacagtgaccaatcatttcagcatctggcttgctacatgtctcagcatcttttattttctcaagatagctaacttttcaaattctatttttctttccctaaggtggagggtaaaaactgtggtttcattaacactgctggtatctcttctcctcttgcttgtaaatgttatcatcataaacacatgtattgttatcttggttgaaggatacaaagtaaatatgtcctacagttctcattcaaacaacaatccacagatttccaggattcctttattcaccaacactatgttcacattcatacccttcacagtgactctgacaattttcctcctgctcatcttctccctgtggaggcatttgaagaagatgcagcatcatgccaagagccccagagaccccagcaccacagcccacattaaggctctgcaaatggtcgtcaccttcctcttcctatacaccattttctttctggcacttgtcatgcaggcttggaaaaatgagattcagtcaaagactgtgttcaacttggtttttgagtcgatagcacttgcttttccttcaggtcactcctgtgtactaattctgggaaactctaagctcagacaggcttttctgaccataatatggtggctgaggtccagttttaatgctgcagaactctcaagtccttag

SEQ ID NO: 52 - protein, 312 aa, R. rattus
MKVTVECALLITLIVEIIIGCLGNGFIAVVNIMDWTKRRRFSLVDQILTALAISRLAFVWSLLTVLVISELHSSLLITRKMLRIINNFWTVTNHFSIWLATCLSIFYFLKIANFSNSIFLSLRWRVKTVVSLTLLVSLLLLLVNVIIINTCIVILVEGYKVNMSYSSHSNNNPQISRIPLFTNTMFTFIPFTVTLTIFLLLIFSLWRHLKKMQHHAKSPRDPSTTAHIKALQMVVTFLFLYTIFFLALVMQAWKNEIQSKTVFNLVFESIALAFPSGHSCVLILGNSKLRQAFLTIIWWLRSSFNAAELSSP

SEQ ID NO: 53 - DNA, 939 bp, r. rattus
atggtggtgacaatgagggctgccctacggctaatgttgataagtactgtaagtctggagctcatcataggaatcttagccaatgtattcatagctctggtgaacatcatagactggattaaaagaggaaagatttctgcagtggataagatctacatgggcctggccatctccaggactgcttttgtattgtcactaatcacagggttcttgatagcatttttggacccagcttcattgggaattggaataatgataagactccttactatatcctggacagtgaccaatcatttcagtgtctggtttgctacatgcctcagcatcttttattttctgaagataaccaatttctcaaacactgttttccttgccctcaaatggaaagttaaaaaagtggtttcggtgacattggtggtgtctctgatcatcttgtttataaacgttatagtcatacacatatacactgatagatttcaagtgaacatggtccagaagtgtggtgcaaataacactttaagagcttacgggctctttctatccatcagcacggtgtttacattcatcccattcacgacatccctgacaatgtttcttctgctcatcttctccctgtggagacacctgaagaccatgcaccacaatgctacaggctccagagatgtcagcaccgtggcccacataaaaggcttgcaaactgtggtcgccttcctgttactatatactgtttttgctatgtcacttttttcacagtctttgagtattgatgctcaacatacaaatcttctttctcactttttacggtgtataggagtggctttcccctcaggccactcctgtgccctgatcctgggaaacaataaactgaggcaggcctctctttctgtgatattttggctgaggtgtaagtacaaacatacagagaatcagggtccctaa

SEQ ID NO: 54 - protein, 312 aa, R. rattus
MVVTMRAALRLMLISTVSLELIIGILANVFIALVNIIDWIKRGKISAVDKIYMGLAISRTAFVLSLITGFLIAFLDPASLGIGIMIRLLTISWTVTNHFSVWFATCLSIFYFLKITNFSNTVFLALKWKVKKVVSVTLVVSLIILFINVIVIHIYTDRFQVNMVQKCGANNTLRAYGLFLSISTVFTFIPFTTSLTMFLLLIFSLWRHLKTMHHNATGSRDVSTVAHIKGLQTVVAFLLLYTVFAMSLFSQSLSIDAQHTNLLSHFLRCIGVAFPSGHSCALILGNNKLRQASLSVIFWLRCKYKHTENQGP

SEQ ID NO: 55 - DNA, 933 bp, r. rattus
atgggtattgtcatagggatcatatgtgcctttattataattgtgcaattcataattgggaatgttgcaaatggattcatagcactggtgaacatcatagactgggtaaagagaagaaaaatctctttagtggatcagatcattactgctttggctatatccaggatagatatgctgtgctctacattcttaattatactaataacttcattgtatccagatctaaatacggctgtgaacatggtaaaaataagcaataatatctggattgttgccaatcatttcagcatctggcttgctacaagcctcagcatcttttatttcctcaagatagctaacttttctaactatgtttttctctgcttaaggtggagacttagcaaagtggtttcagtgacattgctgctctctctggtcctcttgcttatgaatattttaataatgaacatgcatattgatacctggagtgatggattcaaaagaaacgtctcttttggcttcagatcaaagaattgcactctctttttcaaacttgctcttttaatcaacacaacgttcacgtgtgaccccttcactgtgtccatggtggcgtttctgcttctcatcttctccctgtggagacacctgaagaacatgcagtaccatgctaaaggctccagagaccccagcactgccgtgcatataaaggccttacaaatggtggtggtcttcgttctgttctacacatttttctttttgtctcttgccatacaactttggacgtccgagtctctagagaaaaacaatctgttttatgtcactcttataattacttttccttcagtccattcatgtatgctgattctgagaaacagtaaactgaggcaggcatctcttttggtgctgtggtggctgctgtgcagatccaaagacatacagactttggttccctga

SEQ ID NO: 56 - protein, 310 aa, R. rattus
MGIVIGIICAFIIIVQFIIGNVANGFIALVNIIDWVKRRKISLVDQIITALAISRIDMLCSTFLIILITSLYPDLNTAVNMVKISNNIWIVANHFSIWLATSLSIFYFLKIANFSNYVFLCLRWRLSKVVSVTLLLSLVLLLMNILIMNMHIDTWSDGFKRNVSFGFRSKNCTLFFKLALLINTTFTCDPFTVSMVAFLLLIFSLWRHLKNMQYHAKGSRDPSTAVHIKALQMVVVFVLFYTFFFLSLAIQLWTSESLEKNNLFYVTLIITFPSVHSCMLILRNSKLRQASLLVLWWLLCRSKDIQTLVP

SEQ ID NO: 57 - DNA, 963 bp, r. rattus
atggagcattttttgaagagtatatttgatatctccaagaatgtacttccaattattttattcattgaattaataattggaattataggaaatggtttcatggccctggtgcattgcatggactgggttaagagaaaaaaaatgtcattagttaaccaaatcctcaccaccttagcaacctccagaatttgtctgctctggttcatgctattaggtttactaattaccttactggatccagatttagctagtgctagaatgatgatccaggtcgccagtaatctgtggattatagctaaccatatgagcatttggcttgctacatgcctcactgttttttattttctcaagatagccaatttttctagctctctttttctttatctaaagtggagagttgaaaaagtcatttcagttatatttctggtgtcgctggtcttactgtttttaaatatgttactaatgaacttggaaaatgacatgtgtatagctgaatatcatcagataaatatatcgtacagcttcatttaccattaccgtgcagactgcgaaaggcgtgttttaagacttcacattatcatcttgtctgtcccctttgttttgtccctgccaacttttctcctgctcatcttctccctgtggacacatcacaagaagatgcagcagcatgttcaaggacgccgagatgccagcaccacggcccacttcaaagccttgcagaccgtgatcgcctttctcctattatactgtatttttattctgtctatgttactacaattttggaaatatgaattaatgaagaaaccccttttcattttattttgtcatattgtatatggagctttcccttcattccattcatatgtcttgattctgggcgacatgaagctgagacaggcctctctctctgtgctgttgtggctgaaatgcaggccaaattacatagaaacgttagatctctaa

SEQ ID NO: 58 - protein, 320 aa, R. rattus
MEHFLKSIFDISKNVLPIILFIELIIGIIGNGFMALVHCMDWVKRKKMSLVNQILTTLATSRICLLWFMLLGLLITLLDPDLASARMMIQVASNLWIIANHMSIWLATCLTVFYFLKIANFSSSLFLYLKWRVEKVISVIFLVSLVLLFLNMLLMNLENDMCIAEYHQINISYSFIYHYRADCERRVLRLHIIILSVPFVLSLPTFLLLIFSLWTHHKKMQQHVQGRRDASTTAHFKALQTVIAFLLLYCIFILSMLLQFWKYELMKKPLFILFCHIVYGAFPSFHSYVLILGDMKLRQASLSVLLWLKCRPNYIETLDL

SEQ ID NO: 59 - DNA, 789 bp, r. rattus
atgcaacataatttgaagacaatatttgttatctctcacagcacacttacaatcattttattcactgaattagtaactggaattataggaaatgggttcatggccctggtgcactgtatggactggctaaggagaaagaaaatatcattagttaatcaaatcctcactgctttggcaatttccagaatttttcaactctgtttattgtttataagtttagtcatctccttttcatatccagatttaactacaacttcactgataaaagtcacttgtaatctttggattatagtcaaccatttcaacatctggcttgctacatgcctcggtatcttttattttctcaagatatccaatttttctaactctctttttctttatctaaagtggagagttgaaaaagtagttttagttacactgctggtgtcactggtcctactgactttaaatagtttactaattaacttggaaattaacatatgcataaatgaataccaaagaaacataacatacagcttcaattcttattatcatgcacattgtcacaggcagatgttaagccttcatattattttcctgtctgtcccctttgttttgaccctgtcaacttttcccctgatcatcttcttctatgaaggcacatcccccataagatgcgaacacactgtccccgccgacgcgactccaccacaatggacccacttcaaccctctcaaaccccgaccgcctcccacctactcactatacacaaccccccgccacccgaccccccgtatccaccctcagtga

SEQ ID NO: 60 - protein, 262 aa, R. rattus
MQHNLKTIFVISHSTLTIILFTELVTGIIGNGFMALVHCMDWLRRKKISLVNQILTALAISRIFQLCLLFISLVISFSYPDLTTTSLIKVTCNLWIIVNHFNIWLATCLGIFYFLKISNFSNSLFLYLKWRVEKVVLVTLLVSLVLLTLNSLLINLEINICINEYQRNITYSFNSYYHAHCHRQMLSLHIIFLSVPFVLTLSTFPLIIFFYEGTSPIRCEHTVPADATPPQWTHFNPLKPRPPPTYSLYTTPRHPTPRIHPQ

SEQ ID NO: 61 - DNA, 948 bp, r. rattus
atggatggaatcatacagatcatatctgcctttattgtaattatagaaatcataataggatggtttggaaatggatttatagttttggtgaactgcatgcattggatcaagagaagaagaatctctacagtgaatcaaatactcacagccttggctttctccagaatctaccttcttttgacagtattcactgttatattagcatctgtacaatactcaaatatattggtaactagaagggaggtaaaagtgattattttccatttgattaccagcaatcattttagcatgtggcttgctgcatgccttggccttttttattttcttaaaatagctaatttttctaactttatttttgttttcttaaagaagagagttaacaaggtagtttcagggactttgctcatgtctttggtcttcttgtttctaaacactcttctgataaactcatacattgatgcccagatagatgactacagaggatatctgctgtatgatttcacttcaaatatcactgtatcattttacagggttattttagtcattaataactgtattttcacatccataccatttgcactttcacagtcaacttttctcatgctcattttctccctgtggagacattacaagaagatgcaacaacatgcacaaagatgtagagataccctcaccaatgctcacatcaaagtcttgcaaacaatgatcatgtatgtccttctttctgccattttctttctgtttctttcaatgcaaatttggaggaataagttgatggagaacattctttttatcaggttttgtgaaactgttgcagcagtttttccttcaggacactcatgtgtcttgatctggggagacacaaacctgagacagacctttctttctgtgttgtggtggctgaagcacaggttcaccttatgggtccctaaattatattgcagataa

SEQ ID NO: 62 - protein, 315 aa, R. rattus
MDGIIQIISAFIVIIEIIIGWFGNGFIVLVNCMHWIKRRRISTVNQILTALAFSRIYLLLTVFTVILASVQYSNILVTRREVKVIIFHLITSNHFSMWLAACLGLFYFLKIANFSNFIFVFLKKRVNKVVSGTLLMSLVFLFLNTLLINSYIDAQIDDYRGYLLYDFTSNITVSFYRVILVINNCIFTSIPFALSQSTFLMLIFSLWRHYKKMQQHAQRCRDTLTNAHIKVLQTMIMYVLLSAIFFLFLSMQIWRNKLMENILFIRFCETVAAVFPSGHSCVLIWGDTNLRQTFLSVLWWLKHRFTLWVPKLYCR

SEQ ID NO: 63 - DNA, 912 bp, r. rattus
  misc_feature (1)...(912): n = A,T,C or G
atgactttctttttcccagctatttatcacatggtcatcatgacagcagagttcctcatagggactacagtgaatggattccttatcattgtgaactgctatgacttgttcaagagccgagcattcccgatcctgcctaccctcttgatgtgcacagggctgtccagactcgggctgcagataatgctgantgacttaaccttcttctctgtgttctttccatactcttatgaggaaaatatttatagttccaagataatgttcgtttggatgttcttcagctcaattggcctctggtttgccacatgtctttctgtcttttactgcctcaagatttcaggcttcactcagccctggtttctttggctgaaattcagaatttcaaagctcatattttggctgcttctgggcagcttgctggcctctttggggaccgcaactgtgtgtatagaggtaggtttccctttaattgaggatgggtatatcctgaggaacacaagactaaataatagtaatgtcaagctaatgagaaataacaacttactcctcatcaacctgaccttactgcttcccctaactgtgtttgtgatgtgcacctctatgttattcatttctctttacaagcacatgtaccggatgcgaagtgaatctcagaggatgtcaaatgccagaaccgaagcccatataaatgcattaaaaacagtgacatcattcttctgtttctttgtttcttacttcgctgccttcatggcaaatatgacatttagaattccatacagaagtcatcagttctttgtggtgaaggaaatcatggcagcatatcctgccggccactccgtcataatcatcttgagtaactctaagttcaaagacttattcacgagaatgatatgtctgcagaaggaagggtga

SEQ ID NO: 64 - protein, 303 aa, R. rattus
  VARIANT (1)...(303): Xaa = Any Amino Acid
MTFFFPAIYHMVIMTAEFLIGTTVNGFLIIVNCYDLFKSRAFPILPTLLMCTGLSRLGLQIMLXDLTFFSVFFPYSYEENIYSSKIMFVWMFFSSIGLWFATCLSVFYCLKISGFTQPWFLWLKFRISKLIFWLLLGSLLASLGTATVCIEVGFPLIEDGYILRNTRLNNSNVKLMRNNNLLLINLTLLLPLTVFVMCTSMLFISLYKHMYRMRSESQRMSNARTEAHINALKTVTSFFCFFVSYFAAFMANMTFRIPYRSHQFFVVKEIMAAYPAGHSVIIILSNSKFKDLFTRMICLQKEG

SEQ ID NO: 65 - DNA, 960 bp, r. rattus
atggcgcaccccagcaattattggaaacaggatttgttaccactgtccatcttgatcttaacactggtggccactgagtgcaccataggtatcatggcaagtgggatcatcacagttgtgaatgcagtgtcatgggttcagaaaagggcagtttccataactactaggattctgcttcttctgagcgtatccagaataggcctccaaagcatcatcttgatagaaatgacttcctccatattcaacttttcttcttacaacagtgttttatatagagtctcaagggtaagctttgtattcctaaattattgtagcctctggtttgctgctttgcttagtttcttccactttgtgaagattgccaatttttcttaccccctgttcttcaagctaaagtggagaatttctgaattgatgccctggcttctatggctctcggtgtttatttccttcagctccagcatgttcttctgcaatcataaatacactgtgtacaacaacatttctctaagtagcaacatctgcaacttcacaatggaactctatgtcgctgaggccaatgtggtcaatgtggcctttttattcagttttggaatcctcccacctctgaccatgttcattgcaacagctactcttctaattttttctctcaggagacacaccctgcacatgagaaacggtgatgctgactccagaaatccccgagtagaggctcataagcaggccatcaaggaaaccagctgctttctctttctctacatcttatatgcagctgttctgtttctgtccacatccaacatagctgatgccagtctcttctggagtagtgttctcagaatcagtctgcctgtctacccagctggccactcagttttactgattcagagcaaccctggcttaaaaagaacgtggaagcaacttctgtcccaaatccatctgcacttacaaagtagatactga

SEQ ID NO: 66 - protein, 319 aa, R. rattus
MAHPSNYWKQDLLPLSILILTLVATECTIGIMASGIITVVNAVSWVQKRAVSITTRILLLLSVSRIGLQSIILIEMTSSIFNFSSYNSVLYRVSRVSFVFLNYCSLWFAALLSFFHFVKIANFSYPLFFKLKWRISELMPWLLWLSVFISFSSSMFFCNHKYTVYNNISLSSNICNFTMELYVAEANVVNVAFLFSFGILPPLTMFIATATLLIFSLRRHTLHMRNGDADSRNPRVEAHKQAIKETSCFLFLYILYAAVLFLSTSNIADASLFWSSVLRISLPVYPAGHSVLLIQSNPGLKRTWKQLLSQIHLHLQSRY

SEQ ID NO: 67 - DNA, 909 bp, r. rattus
atgctaagtatgctggaaagcatcctcctttctgttgccactagtgaagctatgctgggtattttagggaatatatttattgtacttgtaaactgtacaaactgggtcaggaataagaaactctccaagattaactttattctcactggcttggcaatttccagggtttttaccatatggataataactttagatgcatatacaaaggttttctttctgactacgcttatgcctagcaatctacatgaatgcattagttacatatgggtaattattaaccacctgagtgtctggtttgccacaagcctcagcatcttttatttcctgaagatagcaaacttttcccactacatatttctctggttgaagagaagagctgataaagtttttgtctttctaattggatacttaattataacatggctagcttcctttccactagctgtgacagtgattaaaaatattaaagtgcatcataacaacacatcttggctgatccaactggagaagagagagttacttataaactatgtttttgccaatatggggcccatttccctctttatggtggccgtatttacttgtttcctgttaaccatttccctttggagacacagaaggaggatgcaatccactggatcaaaattcagagatctcaacacagaagttcacgtgaaagccatgaaagttttaatttcatttatcatcctctttatcttatattttatgggtgttctcatagaaacattatgcttgtttctcacagaaaatatacttctctttatttttggcttcactttgtcatccacgtatccctgttgccattcctttatcctaattctaacaagcagggagctgaagcaagcctccatgagggcactgcagagattaaaatgctgtgagacttaa

SEQ ID NO: 68 - protein, 302 aa, R. rattus
MLSMLESILLSVATSEAMLGILGNIFIVLVNCTNWVRNKKLSKINFILTGLAISRVFTIWIITLDAYTKVFFLTTLMPSNLHECISYIWVIINHLSVWFATSLSIFYFLKIANFSHYIFLWLKRRADKVFVFLIGYLIITWLASFPLAVTVIKNIKVHHNNTSWLIQLEKRELLINYVFANMGPISLFMVAVFTCFLLTISLWRHRRRMQSTGSKFRDLNTEVHVKAMKVLISFIILFILYFMGVLIETLCLFLTENILLFIFGFTLSSTYPCCHSFILILTSRELKQASMRALQRLKCCET

SEQ ID NO: 69 - DNA, 960 bp, H. sapiens
atgataacttttctgcccatcattttttccattctaatagtggttatatttgttattggaaattttgctaatggcttcatagcattggtaaattccattgagtgggtcaagagacaaaagatctcctttgttgaccaaattctcactgctctggcggtctccagagttggtttgctctgggtgttattactacattggtatgcaactcagttgaatccagctttttatagtgtagaagtaagaattactgcttataatgtctgggcagtaaccaaccatttcagcagctggcttgctactagcctcagcatgttttatttgctcaggattgccaatttctccaaccttatttttcttcgcataaagaggagagttaagagtgttgttctggtgatactgttggggcctttgctatttttggtttgtcatctttttgtgataaacatggatgagactgtatggacaaaagaatatgaaggaaacgtgacttggaagatcaaattgaggagtgcaatgtaccattcaaatatgactctaaccatgctagcaaactttgtacccctcactctgaccctgatatcttttctgctgttaatctgttctctgtgtaaacatctcaagaagatgcagctccatggcaaaggatctcaagatcccagcaccaaggtccacataaaagctttgcaaactgtgacctcctttcttctgttatgtgccatttactttctgtccatgatcatatcagtttgtaattttgggaggctggaaaagcaacctgtcttcatgttctgccaagctattatattcagctatccttcaacccacccattcatcctgattttgggaaacaagaagctaaagcagatttttctttcagttttgcggcatgtgaggtactgggtgaaagacagaagccttcgtctccatagattcacaagaggggcattgtgtgtcttctag

SEQ ID NO: 70 - protein, 319 aa, H. sapiens
MITFLPIIFSILIVVIFVIGNFANGFIALVNSIEWVKRQKISFVDQILTALAVSRVGLLWVLLLHWYATQLNPAFYSVEVRITAYNVWAVTNHFSSWLATSLSMFYLLRIANFSNLIFLRIKRRVKSVVLVILLGPLLFLVCHLFVINMDETVWTKEYEGNVTWKIKLRSAMYHSNMTLTMLANFVPLTLTLISFLLLICSLCKHLKKMQLHGKGSQDPSTKVHIKALQTVTSFLLLCAIYFLSMIISVCNFGRLEKQPVFMFCQAIIFSYPSTHPFILILGNKKLKQIFLSVLRHVRYWVKDRSLRLHRFTRGALCVF

SEQ ID NO: 71 - DNA, 900 bp, H. sapiens
atgataacttttctgcccatcatattttccattctagtagtggttacatttgttattggaaattttgctaatggcttcatagcgttggtaaattccaccgagtgggtgaagagacaaaagatctcctttgctgaccaaattgtcactgctctggcggtctccagagttggtttgctctgggtgttattattaaattggtattcaactgtgttgaatccagctttttatagtgtagaattaagaactactgcttataatatctgggcagtaaccggccatttcagcaactggcctgctactagcctcagcatattttatttgctcaagattgccaatttctccaaccttatttttcttcgcttaaagaggagagttaagagtgtcattctggtggtgctgttggggcctttgctatttttggcttgtcatctttttgtggtaaacatgaatcagattgtatggacaaaagaatatgaaggaaacatgacttggaagatcaaattgaggcgtgcaatgtacctttcagatacgactgtaaccatgctagcaaacttagtaccctttactgtaaccctgatatcttttctgctgttagtctgttctctgtgtaaacatctcaagaagatgcagctccatggcaaaggatctcaagatcccagtaccaaggtccacataaaagttttgcaaactgtgatctccttcttcttgttatgtgccatttactttgtgtctgtaataatatcagtttggagttttaagaatctggaaaacaaacctgtcttcatgttctgccaagctattggattcagctgttcttcagcccacccgttcatcctgatttggggaaacaagaagctaaagcagacttatctttcagttttgtggcaaatgaggtactga

SEQ ID NO: 72 - protein, 299 aa, H. sapiens [sequence omitted]

SEQ ID NO: 73 - DNA, 900 bp, H. sapiens
atgataacttttctatacatttttttttcaattctaataatggttttatttgttctcggaaactttgccaatggcttcatagcactggtaaatttcattgactgggtgaagagaaaaaagatctcctcagctgaccaaattctcactgctctggcggtctccagaattggtttgctctgggcattattattaaattggtatttaactgtgttgaatccagctttttatagtgtagaattaagaattacttcttataatgcctgggttgtaaccaaccatttcagcatgtggcttgctgctaacctcagcatattttatttgctcaagattgccaatttctccaaccttctttttcttcatttaaagaggagagttaggagtgtcattctggtgatactgttggggactttgatatttttggtttgtcatcttcttgtggcaaacatggatgagagtatgtgggcagaagaatatgaaggaaacatgactgggaagatgaaattgaggaatacagtacatctttcatatttgactgtaactaccctatggagcttcataccctttactctgtccctgatatcttttctgatgctaatctgttctctgtgtaaacatctcaagaagatgcagctccatggagaaggatcgcaagatctcagcaccaaggtccacataaaagctttgcaaactctgatctccttcctcttgttatgtgccattttctttctattcctaatcgtttcggtttggagtcctaggaggctgcggaatgacccggttgtcatggttagcaaggctgttggaaacatatatcttgcattcgactcattcatcctaatttggagaaccaagaagctaaaacacacctttcttttgattttgtgtcagattaggtgctga

SEQ ID NO: 74 - protein, 299 aa, H. sapiens
MITFLYIFFSILIMVLFVLGNFANGFIALVNFIDWVKRKKISSADQILTALAVSRIGLLWALLLNWYLTVLNPAFYSVELRITSYNAWVVTNHFSMWLAANLSIFYLLKIANFSNLLFLHLKRRVRSVILVILLGTLIFLVCHLLVANMDESMWAEEYEGNMTGKMKLRNTVHLSYLTVTTLWSFIPFTLSLISFLMLICSLCKHLKKMQLHGEGSQDLSTKVHIKALQTLISFLLLCAIFFLFLIVSVWSPRRLRNDPVVMVSKAVGNIYLAFDSFILIWRTKKLKHTFLLILCQIRC

SEQ ID NO: 75 - DNA, 930 bp, H. sapiens
atgatgagttttctacacattgttttttccattctagtagtggttgcatttattcttggaaattttgccaatggctttatagcactgataaatttcattgcctgggtcaagagacaaaagatctcctcagctgatcaaattattgctgctctggcagtctccagagttggtttgctctgggtaatattattacattggtattcaactgtgttgaatccaacttcatctaatttaaaagtaataatttttatttctaatgcctgggcagtaaccaatcatttcagcatctggcttgctactagcctcagcatattttatttgctcaagatcgtcaatttctccagacttatttttcatcacttaaaaaggaaggctaagagtgtagttctggtgatagtgttggggtctttgttctttttggtttgtcaccttgtgatgaaacacacgtatataaatgtgtggacagaagaatgtgaaggaaacgtaacttggaagatcaaactgaggaatgcaatgcacctttccaacttgactgtagccatgctagcaaacttgataccattcactctgaccctgatatcttttctgctgttaatctactctctgtgtaaacatctgaagaagatgcagctccatggcaaaggatctcaagatcccagcaccaagatccacataaaagctctgcaaactgtgacctccttcctcatattacttgccatttactttctgtgtctaatcatatcgttttggaattttaagatgcgaccaaaagaaattgtcttaatgctttgccaagcttttggaatcatatatccatcattccactcattcattctgatttgggggaacaagacgctaaagcagacctttctttcagttttgtggcaggtgacttgctgggcaaaaggacagaaccagtcaactccatag

SEQ ID NO: 76 - protein, 309 aa, H. sapiens
MMSFLHIVFSILVVVAFILGNFANGFIALINFIAWVKRQKISSADQIIAALAVSRVGLLWVILLHWYSTVLNPTSSNLKVIIFISNAWAVTNHFSIWLATSLSIFYLLKIVNFSRLIFHHLKRKAKSVVLVIVLGSLFFLVCHLVMKHTYINVWTEECEGNVTWKIKLRNAMHLSNLTVAMLANLIPFTLTLISFLLLIYSLCKHLKKMQLHGKGSQDPSTKIHIKALQTVTSFLILLAIYFLCLIISFWNFKMRPKEIVLMLCQAFGIIYPSFHSFILIWGNKTLKQTFLSVLWQVTCWAKGQNQSTP

SEQ ID NO: 77 - DNA, 957 bp, H. sapiens
atgttaaaggactcagaacaagtgttactaagcctgcatttttttatctgttcaaacatgatgtgttttctgctcatcatttcatcaattctggtagtgtttgcatttgttcttggaaatgttgccaatggcttcatagccctagtaaatgtcattgactgggttaacacacgaaagatctcctcagctgagcaaattctcactgctctggtggtctccagaattggtttactctgggtcatgttattcctttggtatgcaactgtgtttaattctgctttatatggtttagaagtaagaattgttgcttctaatgcctgggctgtaacgaaccatttcagcatgtggcttgctgctagcctcagcatattttgtttgctcaagattgccaatttctccaaccttatttctctccacctaaagaagagaattaagagtgttgttctggtgatactgttggggcccttggtatttctgatttgtaatcttgctgtgataaccatggatgagagagtgtggacaaaagaatatgaaggaaatgtgacttggaagatcaaattgaggaatgcaatacacctttcaagcttgactgtaactactctagcaaacctcataccctttactctgagcctaatatgttttctgctgttaatctgttctctttgtaaacatctcaagaagatgcggctccatagcaaaggatctcaagatcccagcaccaaggtccatataaaagctttgcaaactgtgacctccttcctcatgttatttgccatttactttctgtgtataatcacatcaacttggaatcttaggacacagcagagcaaacttgtactcctgctttgccaaactgttgcaatcatgtatccttcattccactcattcatcctgattatgggaagtaggaagctaaaacagacctttctttcagttttgtggcagatgacacgctga

SEQ ID NO: 78 - protein, 318 aa, H. sapiens
MLKDSEQVLLSLHFFICSNMMCFLLIISSILVVFAFVLGNVANGFIALVNVIDWVNTRKISSAEQILTALVVSRIGLLWVMLFLWYATVFNSALYGLEVRIVASNAWAVTNHFSMWLAASLSIFCLLKIANFSNLISLHLKKRIKSVVLVILLGPLVFLICNLAVITMDERVWTKEYEGNVTWKIKLRNAIHLSSLTVTTLANLIPFTLSLICFLLLICSLCKHLKKMRLHSKGSQDPSTKVHIKALQTVTSFLMLFAIYFLCIITSTWNLRTQQSKLVLLLCQTVAIMYPSFHSFILIMGSRKLKQTFLSVLWQMTR

SEQ ID NO: 79 - DNA, 930 bp, H. sapiens
atgataacttttctgcccatcattttttccattctaatagtggttacatttgtgattggaaattttgctaatggcttcatagcattggtaaattccattgagtggttcaagagacaaaagatctcttttgctgaccaaattctcactgctctggcagtctccagagttggtttactctgggtattagtattaaattggtatgcaactgagttgaatccagcttttaacagtatagaagtaagaattactgcttacaatgtctgggcagtaatcaaccatttcagcaactggcttgctactagcctcagcatattttatttgctcaagattgccaatttctccaaccttatttttcttcacttaaagaggagagttaagagtgttgttctggtgatactattggggcctttgctatttttggtttgtcatctttttgtgataaacatgaatcagattatatggacaaaagaatatgaaggaaacatgacttggaagatcaaactgaggagtgcaatgtacctttcaaatacaacggtaaccatcctagcaaacttagttcccttcactctgaccctgatatcttttctgctgttaatctgttctctgtgtaaacatctcaaaaagatgcagctccatggcaaaggatctcaagatcccagcatgaaggtccacataaaagctttgcaaactgtgacctccttcctcttgttatgtgccatttactttctgtccataatcatgtcagtttggagttttgagagtctggaaaacaaacctgtcttcatgttctgcgaagctattgcattcagctatccttcaacccacccattcatcctgatttggggaaacaagaagctaaagcagacttttctttcagttttgtggcatgtgaggtactgggtgaaaggagagaagccttcatcttcatag

SEQ ID NO: 80 - protein, 309 aa, H. sapiens
MITFLPIIFSILIVVTFVIGNFANGFIALVNSIEWFKRQKISFADQILTALAVSRVGLLWVLVLNWYATELNPAFNSIEVRITAYNVWAVINHFSNWLATSLSIFYLLKIANFSNLIFLHLKRRVKSVVLVILLGPLLFLVCHLFVINMNQIIWTKEYEGNMTWKIKLRSAMYLSNTTVTILANLVPFTLTLISFLLLICSLCKHLKKMQLHGKGSQDPSMKVHIKALQTVTSFLLLCAIYFLSIIMSVWSFESLENKPVFMFCEAIAFSYPSTHPFILIWGNKKLKQTFLSVLWHVRYWVKGEKPSSS

SEQ ID NO: 81 - DNA, 930 bp, H. sapiens
atgacaacttttatacccatcattttttccagtgtggtagtggttctatttgttattggaaattttgctaatggcttcatagcattggtaaattccattgagcgggtcaagagacaaaagatctcttttgctgaccagattctcactgctctggcggtctccagagttggtttgctctgggtattattattaaattggtattcaactgtgtttaatccagctttttatagtgtagaagtaagaactactgcttataatgtctgggcagtaaccggccatttcagcaactggcttgctactagcctcagcatattttatttgctcaagattgccaatttctccaaccttatttttcttcacttaaagaggagagttaagagtgtcattctggtgatgctgttggggcctttactatttttggcttgtcaactttttgtgataaacatgaaagagattgtacggacaaaagaatatgaaggaaacttgacttggaagatcaaattgaggagtgcagtgtacctttcagatgcgactgtaaccacgctaggaaacttagtgcccttcactctgaccctgctatgttttttgctgttaatctgttctctgtgtaaacatctcaagaagatgcagctccatggtaaaggatctcaagatcccagcaccaaggtccacataaaagctttgcaaactgtgatctttttcctcttgttatgtgccgtttactttctgtccataatgatatcagtttggagttttgggagtctggaaaacaaacctgtcttcatgttctgcaaagctattagattcagctatccttcaatccacccattcatcctgatttggggaaacaagaagctaaagcagacttttctttcagttttgcggcaagtgaggtactgggtgaaaggagagaagccttcatctccatag

SEQ ID NO: 82 - protein, 309 aa, H. sapiens
MTTFIPIIFSSVVVVLFVIGNFANGFIALVNSIERVKRQKISFADQILTALAVSRVGLLWVLLLNWYSTVFNPAFYSVEVRTTAYNVWAVTGHFSNWLATSLSIFYLLKIANFSNLIFLHLKRRVKSVILVMLLGPLLFLACQLFVINMKEIVRTKEYEGNLTWKIKLRSAVYLSDATVTTLGNLVPFTLTLLCFLLLICSLCKHLKKMQLHGKGSQDPSTKVHIKALQTVIFFLLLCAVYFLSIMISVWSFGSLENKPVFMFCKAIRFSYPSIHPFILIWGNKKLKQTFLSVLRQVRYWVKGEKPSSP

SEQ ID NO: 83 - DNA, 969 bp, H. sapiens
atgctcttacaggcaatgggtggtgtcataaagagcatatttacattcgttttaattgtggaatttataattggaaatttaggaaatagtttcatagcactggtgaactgtattgactgggtcaagggaagaaagatctcttcggttgatcggatcctcactgctttggcaatctctcgaattagcctggtttggttaatattcggaagctggtgtgtgtctgtgtttttcccagctttatttgccactgaaaaaatgttcagaatgcttactaatatctggacagtgatcaatcattttagtgtctggttagctacaggcctcggtactttttattttctcaagatagccaatttttctaactctatttttctctacctaaagtggagggttaaaaaggtggttttggtgctgcttcttgtgacttcggtcttcttgtttttaaatattgcactgataaacatccatataaatgccagtatcaatggatacagaagaaacaagacttgcagttctgattcaagtaactttacacgattttccagtcttattgtattaaccagcactgtgttcattttcataccctttactttgtccctggcaatgtttcttctcctcatcttctccatgtggaaacatcgcaagaagatgcagcacactgtcaaaatatccggagacgccagcaccaaagcccacagaggagttaaaagtgtgatcactttcttcctactctatgccattttctctctgtcttttttcatatcagtttggacctctgaaaggttggaggaaaatctaattattctttcccaggtgatgggaatggcttatccttcatgtcactcatgtgttctgattcttggaaacaagaagctgagacaggcctctctgtcagtgctactgtggctgaggtacatgttcaaagatggggagccctcaggtcacaaagaatttagagaatcatcttga

SEQ ID NO: 84 - protein, 322 aa, H. sapiens [sequence omitted]

SEQ ID NO: 85 - DNA, 1179 bp, H. sapiens
atggcttcagccagccgcggcaacctacctgggttgcctttcactctgtggaagctttgtcctttctctcttctcaataaaccttgctgttgctcactctttgggtccacaccatctttaagagcagcaacactcaccgtgaaggtcagtggctcccttttgaagtcagcgagaccacgaacccacctgcaggaaccaattccggacacaacaccagcatttaaaaaaattttttttgtctgttcagacatgataacttttctacccatcattttttccagtctggtagtggttacatttgttattggaaattttgctaatggcttcatagcactggtaaattccattgagtggttcaagagacaaaagatctcctttgctgaccaaattctcactgctctggcggtctccagagttggtttgctctgggtattattattaaactggtattcaactgtgttgaatccagcttttaatagtgtagaagtaagaactactgcttataatatctgggcagtgatcaaccatttcagcaactggcttgctactaccctcagcatattttatttgctcaagattgccaatttctccaactttatttttcttcacttaaagaggagagttaagagtgtcattctggtgatgttgttggggcctttgctatttttggcttgtcatctttttgtgataaacatgaatgagattgtgcggacaaaagaatttgaaggaaacatgacttggaagatcaaattgaagagtgcaatgtacttttcaaatatgactgtaaccatggtagcaaacttagtacccttcactctgaccctactatcttttatgctgttaatctgttctttgtgtaaacatctcaagaagatgcagctccatggtaaaggatctcaagatcccagcaccaaggtccacataaaagctttgcaaactgtgatctccttcctcttgttatgtgccatttactttctgtccataatgatatcagtttggagttttggaagtctggaaaacaaacctgtcttcatgttctgcaaagctattagattcagctatccttcaatccacccattcatcctgatttggggaaacaagaagctaaagcagacttttctttcagttttttggcaaatgaggtactgggtgaaaggagagaagacttcatctccatag

SEQ ID NO: 86 - protein, 309 aa, H. sapiens
MITFLPIIFSSLVVVTFVIGNFANGFIALVNSIEWFKRQKISFADQILTALAVSRVGLLWVLLLNWYSTVLNPAFNSVEVRTTAYNIWAVINHFSNWLATTLSIFYLLKIANFSNFIFLHLKRRVKSVILVMLLGPLLFLACHLFVINMNEIVRTKEFEGNMTWKIKLKSAMYFSNMTVTMVANLVPFTLTLLSFMLLICSLCKHLKKMQLHGKGSQDPSTKVHIKALQTVISFLLLCAIYFLSIMISVWSFGSLENKPVFMFCKAIRFSYPSIHPFILIWGNKKLKQTFLSVFWQMRYWVKGEKTSSP

SEQ ID NO: 87 - DNA, 951 bp, H. sapiens
atggagcatcttttgaagagaacatttgatatcactgagaacatacttcaaattattttattcattgaattaataattggacttataggaaatggattcacagccttggtgcactgcatggattgggttaagagaaaaaaaatgtcattagttaataaaatcctcaccgctttggcaacttctagaattttcctgctctggttcatgctagtaggttttccaattagctcactgtacccatatttagttactactagactgatgatacagttcactagtactctatggactatagctaaccatattagtgtctggtttgctacatgcctcagtgtcttttattttctcaagatagccaatttttctaattctccttttctctatctaaagaggagagttgaaaaagtagtttcagttacattactggtatctctggtcctcttgtttttaaatattttactacttaatttggaaattaatgtgtgtataaatgaatatcatcaaataaacacatcatatatcttcatttcttattaccatttaagttgtcaaattcaggtgttaggaagtcacattattttcctgtttgcccccgttgttttgtccctgtcaacttttctcctgctcatcttctccctgtggacacatcacaagaggatgcagcagcatgttcagggagacagagatgccagaactatggcccacttcaaagccttgcaaaccgtgattgcctttcttctactacactccatttttatcctgtcactgttactacaattttggatccatgaattaaggaagaaacctcctttcgttgcattttgtcaggttgtatatatagcttttccttcattccattcatatgtcttgattctgagagacagaaagctgagacatgcctctctttctgtgttgtcatggctgaaatgcaggccaaattatgtggaataa

SEQ ID NO: 88 - protein, 316 aa, H. sapiens
MEHLLKRTFDITENILQIILFIELIIGLIGNGFTALVHCMDWVKRKKMSLVNKILTALATSRIFLLWFMLVGFPISSLYPYLVTTRLMIQFTSTLWTIANHISVWFATCLSVFYFLKIANFSNSPFLYLKRRVEKVVSVTLLVSLVLLFLNILLLNLEINVCINEYHQINTSYIFISYYHLSCQIQVLGSHIIFLFAPVVLSLSTFLLLIFSLWTHHKRMQQHVQGDRDARTMAHFKALQTVIAFLLLHSIFILSLLLQFWIHELRKKPPFVAFCQVVYIAFPSFHSYVLILRDRKLRHASLSVLSWLKCRPNYVE

SEQ ID NO: 89 - DNA, 1002 bp, H. sapiens
atgttgactctaactcgcatccgcactgtgtcctatgaagtcaggagtacatttctgttcatttcagtcctggagtttgcagtggggtttctgaccaatgccttcgttttcttggtgaatttttgggatgtagtgaagaggcaggcactgagcaacagtgattgtgtgctgctgtgtctcagcatcagccggcttttcctgcatggactgctgttcctgagtgctatccagcttacccacttccagaagttgagtgaaccactgaaccacagctaccaagccatcatcatgctatggatgattgcaaaccaagccaacctctggcttgctgcctgcctcagcctgctttactgctccaagctcatccgtttctctcacaccttcctgatctgcttggcaagctgggtctccaggaagatctcccagatgctcctgggtattattctttgctcctgcatctgcactgtcctctgtgtttggtgcttttttagcagacctcacttcacagtcacaactgtgctattcatgaataacaatacaaggctcaactggcagattaaagatctcaatttattttattcctttctcttctgctatctgtggtctgtgcctcctttcctattgtttctggtttcttctgggatgctgactgtctccctgggaaggcacatgaggacaatgaaggtctataccagaaactctcgtgaccccagcctggaggcccacattaaagccctcaagtctcttgtctcctttttctgcttctttgtgatatcatcctgtgttgccttcatctctgtgcccctactgattctgtggcgcgacaaaataggggtgatggtttgtgttgggataatggcagcttgtccctctgggcatgcagccatcctgatctcaggcaatgccaagttgaggagagctgtgatgaccattctgctctgggctcagagcagcctgaaggtaagagccgaccacaaggcagattcccggacactgtgctga

SEQ ID NO: 90 - protein, 333 aa, H. sapiens
MLTLTRIRTVSYEVRSTFLFISVLEFAVGFLTNAFVFLVNFWDVVKRQALSNSDCVLLCLSISRLFLHGLLFLSAIQLTHFQKLSEPLNHSYQAIIMLWMIANQANLWLAACLSLLYCSKLIRFSHTFLICLASWVSRKISQMLLGIILCSCICTVLCVWCFFSRPHFTVTTVLFMNNNTRLNWQIKDLNLFYSFLFCYLWSVPPFLLFLVSSGMLTVSLGRHMRTMKVYTRNSRDPSLEAHIKALKSLVSFFCFFVISSCVAFISVPLLILWRDKIGVMVCVGIMAACPSGHAAILISGNAKLRRAVMTILLWAQSSLKVRADHKADSRTLC

(19) United States
(12) Patent Application Publication
(10) Pub. No.: US 2007/0059688 A1
(43) Pub. Date: Mar. 15, 2007

(54) Gastrointestinal chemosensory receptors

(76) Inventor: Juan E Rozengurt, (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/492,470

(22) PCT Filed: Oct. 11, 2002
(86) PCT No.: PCT/US02/32664

§ 371 Date: Oct. 4, 2006

Related U.S. Application Data
(60) Provisional application No. 60/328,993, filed on Oct. 12, 2001.

(51) Int. Cl. C12Q 1/68; G01N 33/53; C07H 21/04; C12P 21/06; C07K 14/705

(52) U.S. Cl. 435/6; 435/7.1; 435/69.1; 435/320.1; 435/325; 530/350; 536/23.5

(57) ABSTRACT

This invention provides isolated nucleic acid and amino acid sequences of gastrointestinal endocrine cell specific G-protein coupled receptors, methods of detecting such receptors, and methods of screening for ligands of such receptors. Furthermore, this invention demonstrates that SrC-1 enteroendocrine cells express multiple bitter taste receptors as well as a-subunits of G proteins that mediate taste signal transduction and respond to bitter taste compounds initiating changes in intracellular calcium concentration. Given that at present there are no cultured cell model system to determine the functional effects of taste receptor-mediated signaling, our findings identify STC-1 cells as a cell model for studying taste-mediated signal transduction.

### GOVERNMENT INTERESTS

This invention was made with government support under Grant No. DK17294, awarded by the National Institute of Health. The government has certain rights in this invention.

### FIELD OF THE INVENTION

The invention provides isolated nucleic acid and amino acid sequences of GI endocrine cell specific G-protein coupled receptors, methods of detecting such nucleic acids and receptors, and models of screening for native and artificial ligands of GI-specific G-protein coupled receptor.

### BACKGROUND OF THE INVENTION

The gustatory system has been selected during evolution to detect nutritive and beneficial compounds as well as harmful or toxic substances (Herness et. al. Annu. Rev. Physiol. 61, 873-900, (1999)). In particular, bitter taste has evolved as a central warning signal against the ingestion of potentially toxic substances (Glendinning et. al. Behav. Neurosci. 113, 840-854, (1999)). Recently, a large family of bitter taste receptors (T2Rs) expressed in specialized neuroepithelial taste receptor cells organized within taste buds in the tongue has been identified in humans and rodents (Chandrashekar et. al. Cell 100, 703-711, (2000); Adler et. al. Cell 100, 693-702, (2000); Matsunami et. al. Nature (London) 404, 601-604, (2000)). These putative taste receptors, which belong to the guanine nucleotide-binding regulatory protein (G protein)-coupled receptor (GPCR) superfamily characterized by seven putative transmembrane domains, are distantly related to V1R vomeronasal receptors and opsins (Adler et. al. Cell 100, 693-702, (2000)). Genetic and biochemical evidence indicate that specific Gα, subunits, gustducin (Gα_{gust}) and transducin (Gαₜ), mediate bitter and sweet gustatory signals in the taste buds of the lingual epithelium (Ruiz-Avila et. al. Nature (London) 376, 80-85, (1995); Wong et. al. Nature (London) 381, 796-800, (1996); Ming et. al. Proc. Natl. Acad. Sci. USA 96, 9903-9908, (1999)).

Outside the tongue, expression of Gα_{gust} has been also localized to gastric (Hoefer et. al. Proc. Natl. Acad. Sci. USA 93, 6631-6634, (1996)) and pancreatic cells (Hoefer et. al. (1998) Histochem. Cell. Biol. 110, 303-309, (1998)), suggesting that a taste-sensing mechanism may also exist in the gastrointestinal (GI) tract. However, not all cells that express Gα_{gust} also co-express members of the T2R family of receptors (Adler et. al. Cell 100, 693-702, (2000)). For example, most Gα_{gust}-positive taste receptor cells in the lingual fungiform papillae are T2R-negative, implying that Gα_{gust} could also mediate signaling through other receptors (Wong et. al. Nature (London) 381, 796-800, (1996)). In order to establish that the gastric and intestinal mucosa play a role in molecular sensing and to unravel the signaling mechanisms involved, it is of critical importance to identify taste receptor gene transcripts in the lining of the stomach or intestine.

In addition to the gustatory system, the olfactory system must discriminate among thousands of odors comprised of chemically divergent structures (odorants). It is generally accepted that the primary olfactory sensory receptor neurons are located in the olfactory epithelium, where they are in direct contact with inhaled odorants. Odorant signal transduction is initiated when odorants interactwith specific GPCRs located in the surface of olfactory sensory neurons and activate a specific heterotrimeric G protein, Gα_{olf}, which promotes the accumulation of cAMP (Ronnett et.al. Annu. Rev. Physiol. 64:189-222, (2002)). However, several studies indicate that receptors closely related to olfactory receptors genes may be expressed in tissues other than the olfactory epithelium. This finding suggests that there may be alternative biological roles for this family of chemosensory receptors.

Expression of various olfactory receptors was reported in human and murine erythroid cells (Feingold et.al. Genomics 61:15-23, (1999)), developing rat heart (Drutel e.al. Recept. Channel 3:33-40, (1995)), avian notochord (Nef et. al. Proc. Natl. Acad. Sci. USA 94:476-671, 1997)) and lingual epithelium (Abe et. al. FEBS Lett. 316:253-56, (1993)). The best case for the existence of olfactory receptors is the finding that genes related to mammalian olfactory GPCRs are transcribed in testes and expressed on the surface of mature spermatozoa, suggesting a possible role for olfactory receptors in sperm chemotaxis (Walensky et. al. J. Biol. Chem. 273:9378-87, (1998)). We considered the possibility that, in addition of taste receptors, enteroendocrine cells or other cell types in the gastrointestinal tract can express odorant receptors and the corresponding signal transducer, Gα_{olf}.

Molecular sensing of the luminal contents of the GI tract not only regulates motility, release of GI hormones, and pancreatobiliary secretion, but it is also responsible for the detection of ingested drugs and toxins thereby initiating responses critical for survival. The enteroendocrine cells, which produce and release more than 20 identified hormones, are thought to play a critical role in the integration and coordination of these physiological responses. (Furness et. al. Am. J. Physiol. 277, G922-G928, (1999)). Although these fundamental control systems have been known for decades, the initial molecular recognition events that sense the chemical composition of the luminal contents have remained elusive.

In view of the importance of chemical sensing in food intake, digestion and poison rejection, the expression of taste and olfactory receptors is of interest. The identification and isolation of chemical sensing receptors (including taste ion channels), and signaling molecules would allow for the pharmacological and genetic modulation of taste transduction pathways. For example, availability of receptor and channel molecules would permit the screening for high affinity agonists, antagonists, inverse agonists, and modulators of chemosensory cell activity. Such compounds could then be used in the pharmaceutical and food industries to customize taste.

### SUMMARY OF THE INVENTION

The present invention identifies a family of taste-sensing receptors in the stomach and intestine that perceive chemical components of ingested substances including drugs and toxins has important implications for understanding molecular sensing in the GI tract and for developing novel therapeutic compounds that modify the function of these receptors in the gut. Such theraeutic compounds have have a functional effect on the release of peptide hormones and neurotransmitters, which are known regulators of gastrointestinal motility and reflex, mucosal growth, ion and enzyme secretion, satiety and appetite. Therapeutic compounds may also result in changes in receptor phosphorylation, internalization, and redistribution, which would modify taste sensitivity and adaptation.

In one aspect, the present invention provides an isolated nucleic acid encoding a gastrointestinal taste transduction G-protein coupled receptor, referred herein as GT2R, the receptor comprising at least 50% amino acid identity, usually greater than 60% sequence identity and may have 70%, 80% or 90% identity to an amino acid sequence of selected from the group consisting of (a) mouse GT2R: SEQ ID NOS:2, 4, 6, 8, 10, 12, 14, 16, 18, 20, 22, 24, 26, 28, 30, or from the group consisting of (b) rat GT2R: SEQ ID NOS: 32, 34, 36, 38, 40, 42, 44, 46, 48, 50, 52, 54, 56, 58, 60, 62, 64, 66, 68, or from the group consisting of (c) human GT2R SEQ ID NOS:70, 72, 74, 76, 78, 80, 82, 84, 86, 88, 90.

In one aspect, the present invention provides an isolated polypeptide comprising a transmembrane domain of a sensory transduction G-protein coupled receptor, the transmembrane domain comprising at least 60% amino acid sequence identity, usually greater than 70% identity, and may have 80% or 90% identity to a transmembrane domain sequence selected from the group consisting of (a) mouse GT2R: SEQ ID NOS:2, 4, 6, 8, 10, 12, 14, 16, 18, 20, 22, 24, 26, 28, 30, or from the group consisting of (b) rat GT2R: SEQ ID NOS: 32, 34, 36, 38, 40, 42, 44, 46, 48, 50, 52, 54, 56, 58, 60, 62, 64, 66, 68, or from the group consisting of (c) human GT2R SEQ ID NOS:70, 72, 74, 76, 78, 80, 82, 84, 86, 88, 90.

In another aspect, the present invention provides an expression vector comprising a nucleic acid encoding a polypeptide comprising greater than about 50% amino acid sequence identity to an amino acid sequence selected from the group consisting of (a) mouse GT2R: SEQ ID NOS:2, 4, 6, 8, 10, 12, 14, 16, 18, 20, 22, 24, 26, 28, 30, or from the group consisting of (b) rat GT2R: SEQ ID NOS: 32, 34, 36, 38, 42, 44, 46, 48, 50, 52, 54, 56, 58, 60, 62, 64, 66, 68, or from the group consisting of (c) human GT2R SEQ ID NOS:70, 72, 74, 76, 78, 80, 82, 84, 86, 88, 90.

In another aspect, the present invention provides a host cell line STC-1, which expresses endogenous GT2R comprising a sequence selected from the group consisting of SEQ ID NO:l, SEQ ID NO:3, SEQ ID NO:5, SEQ ID NO:7, SEQ ID NO:9, SEQ ID NO:11, SEQ ID NO:13. In another aspect, the present invention provides a host cell line STC-1, which can be transfected with the expression vector containing recombinant GT2R comprising a sequence selected from the group consisting of (a) mouse GT2R SEQ ID NOS:15, 17, 19, 21, 23, 25, 27 29, or from the group consisting of (b) rat GT2R SEQ ID NOS:31, 33, 35, 37, 41, 43, 45, 47, 49, 51, 53, 55, 57, 59, 61, 63, 65, 67, or from the group consisting of (c) human GT2R SEQ ID NOS: 69, 71, 73, 75, 77, 79, 81, 83, 85, 87, 89.

In another aspect, the present invention provides a method for identifying a compound that modulates taste signaling in gastrointestinal chemosensory cells, the method comprising the steps of: (i) contacting the compound with a taste transduction G-protein coupled receptor polypeptide, wherein the polypeptide is expressed in STC-1 cells, the polypeptide comprising greater than 60% amino acid identity to a sequence selected from the group consisting of SEQ ID NO:2, SEQ ID NO:4, or SEQ ID NO:6; and SEQ ID NO:8, SEQ ID NO:10, SEQ ID NO:12, SEQ ID NO:14 (ii) determining the functional effects of the compound on the polypeptides.

In one embodiment, the polypeptide is a taste-sensing G-protein coupled receptor, the receptor comprising greater than about 50% amino acid identity, usually at least 60% sequence identity and may have 70%, 80% or 90% identity to a polypeptide selected from the group of (a) mouse GT2R SEQ ID NOS:2, 4, 6, 8, 10, 12, 14, 16, 18, 20, 22, 24, 26, 28, 30, or from the group of (b) rat GT2R SEQ ID NOS:32, 34, 36, 38, 42, 44, 46, 48, 50, 52, 54, 56, 58, 60, 62, 64, 66, 68, or from the group of (c) human GT2R SEQ ID NOS:70, 72, 74, 76, 78, 80, 82, 84, 86, 88, 90. In another embodiment, the polypeptide has G-protein coupled receptor activity. In another embodiment, the functional effect is determined by measuring changes in intracellular cAMP, IP3, or Ca²⁺. In another embodiment, the functional effect is a chemical effect. In another embodiment, the functional effect is determined by measuring binding of the compound to the binding domains. In another embodiment, the polypeptide is recombinant. In another embodiment, the polypeptide is from a mouse, a rat, or a human. In another embodiment, the polypeptide is expressed in a gastric gland, an intestinal gland, a cell line or cell membrane. In another embodiment, the cell is a eukaryotic cell.

### DESCRIPTION OF THE DRAWINGS

FIG. 1 demonstrates expression of Gαₜ₋₂, Gα_{gust}, and members of the GT2R family in STC-1 cells. A: RT-PCR analysis for the a subunits of Gαₜ₋₂ and Gα_{gust} was performed on poly A⁺ RNA isolated from STC-1 cells. PCR products with the predicted size (indicated by an arrow) were subcloned and sequenced to confirm their identity. B: Immunoblot analysis for Gαₜ₋₂ and Gα_{gust} was performed on total protein extracts prepared from STC-1 cells. Normal or pre-absorbed Gαₜ₋₂ and Gα_{gust}-speciflc antibodies were used to detect the presence of their respective Gα, subunits in total protein extracts electrophoresed and blotted onto the nitrocellulose membrane. C: RT-PCR analysis using T2R-specific primers was performed on the same cDNAs used in experiments described in section A. PCR products corresponding to the predicted mT2Rs were subcloned and sequenced to confirm their relatedness to published rat and mouse sequences.

FIG. 2 illustrates predicted amino acid sequences of mouse GT2R homologues isolated from STC-1 cells. Complete sequences were deduced from cDNA clones of STC-1 generated by RT-PCR using degenerate or specific primers and from genomic clones isolated from the mouse BAC genomic DNA libraries. ClustalW alignment for multiple sequences and homology analysis were performed using MacVector software (ver. 7.1, Accelrys Inc.).

FIG. 3 shows tissue distribution of mT2R19 transcripts, which is a known mouse ortholog of human T2R1 and rat T2R1, in mouse upper GI tract. RT-PCR using mT2R19-specific primers was performed on cDNAs prepared from various mouse tissues. PCR products were separated on 1% agarose gel containing EtBr and the identity of the predicted mT2R19 cDNA fragment (698 bp) was confirmed by DNA sequencing. A: antrum; F: fundus; D: duodenum; I: ileum; J: jejunum; C: colon; L: liver; H: heart; K: kidney; and T: tongue. As a control, β-actin from the respective samples was amplified and shown in the bottom panel.

FIGS. 4A-4D demonstrate immunostaining of Gαₜ₋₂ and Gα_{gust} in mouse fundus (A and B) and antrum (C and D). FIG. 4A—Immunostaining with antibody against Gαₜ₋₂. The base of the fundic glands is rich in positively stained cells (arrows) (×40). Insert: Higher power (×100) of a gland showing that the stained cell is round, with a central or slightly eccentric nucleus and pale cytoplasm. FIG. 4B—A consecutive section stained with antibody against Gα_{gust}. There are no positive cells at this portion of the gland (×40). FIG. 4C—Antral mucosa immunostained with antibody against Gαₜ₋₂. There are no positive cells (×40). FIG. 4D—A consecutive section stained with antibody against Gα_{gust}. Arrows point at some of the many positively stained cells (×40). Insert: Higher power (×100) of a Gα_{gust}-positive cell exhibiting an elongated shape with a luminal pole and a projection towards the basement membrane.

FIGS. 5A-5D show the immunostaining of Gα_{gust} in lingual taste buds (A and B) and gastric antrum (C and D). FIG. 5A—Immunostaining of lingual epithelium with antibody against Gxgust reveals the presence of Gα_{gust}-positive cells in the taste bud. FIG. 5B—A consecutive section of the lingual epithelium immunostained with antibody against Gα_{gust} but incubated in the presence of the immunizing peptide. FIG. 5C—Antral mucosa immunostained with antibody against Gα_{gust}. FIG. 5D—A consecutive section immunostained with antibody against Gα_{gust} but incubated in the presence the immunizing peptide. Note that the addition of the immunogenic peptide completely blocked the staining of either the cells in the taste bud or the cells of the gastric mucosa. In contrast, incubation of the antibody in the presence of structurally unrelated peptides corresponding to the α subunit of Gα_{olf} or to a region of extracellular-signal-regulated kinase did not reduce the immunostaining of the gastric epithelial cells. The antibody used was an affinity-purified rabbit polyclonal antibody raised against a peptide corresponding to amino acids 93-112 of Gα_{gust}, a highly divergent sequence in the rat protein [Gα_{gust} (1-20); Santa Cruz Biotechnology]. Gα_{gust} (1-20) reacts specifically with the a subunit of gustducin of mouse, rat, and human cell origin as shown by Western blotting and immunohistochemistry but does not cross-react with other Gα subunits, including rod (Gαₜ₋₁) or cone (Gαₜ₋₂) transducins (Santa Cruz Biotechnology). (Magnifications: A and B, ×40; C and D, ×20.)

FIGS. 6A-6D show the immunostaining of Gαₜ₋₂ in mouse retina (A and B) and stomach fundus (C and D). FIG. 6A—Immunostaining with antibody against Gαₜ₋₂ in the retina. FIG. 6B—A consecutive section of retina was immunostained with antibody against Gαₜ₋₂ but incubated in the presence of the immunizing peptide. FIG. 6C—Immunostaining with antibody against Gαₜ₋₂ in the base of the fundic glands. FIG. 6D—A consecutive section of base of the fundic glands were immunostained with antibody against Gαₜ₋₂ but incubated in the presence of the immunizing peptide. 1, Pigment epithelium; 2, photoreceptor (cones and rods) layer. Note that the addition of the immunogenic peptide completely blocked the staining of either the cones of the retina or the cells of the gastric mucosa. In contrast, incubation of the antibody in the presence of structurally unrelated peptides corresponding to the α subunit of Golf or to a region of extracellular-signal-related kinase did not reduce the immunostaining of the gastric epithelial cells. In these experiments, the antibody used was an affinity-purified rabbit polyclonal antibody against a-transducin-2 [Gαₜ₋₂ (1-20); Santa Cruz Biotechnology] that reacts with mouse, rat, and human cell origin as shown by Western blotting and immunohistochemistry but does not cross-react with other Gα subunits including Gαₜ₋₁ (Santa Cruz Biotechnology). (Magnifications: ×20)

FIG. 7 shows the expression of Gαₜ and Gα_{gust} in rat GI tissues and in a rat gastric endocrine cell cDNA library. Consensus primers to amplify the α subunits of Gαₜ₋₂ and Gα_{gust} by PCR were designed based on the published rat, mouse and human Gα sequences. PCR amplification reactions were performed on reverse transcribed mRNA from rat antrum (A), fundus (F), and duodenum (D), and on cDNA from a gastric endocrine cell library. The predicted sizes of the PCR products of Gαₜ₋₂ and Gα_{gust} are 340 bp and 332 bp, respectively.

FIG. 8 shows the expression of known rT2R family members in rat GI tract. RT-PCR was performed using rat-specific primers for each of the eleven rT2R subtypes on poly A⁺ mRNA isolated from rat antral, fundic, duodenal mucosa and IEC-6 cells, and on cDNAs from a rat gastric endocrine cell cDNA library. PCR products were separated on 1% agarose gel containing ethidium bromide and products with the predicted size for each rT2R subtype were subcloned and sequenced to verify their identity. Control transcript β-actin from the respective sample is shown in the adjacent lanes.

FIG. 9 illustrates response of STC-1 cells to bitter tastant molecules with an increase in intracellular calcium concentration. The [Ca²⁺]ᵢ of individual cells was measured before and after exposure to single concentrations of bitter tastants. Top panel. Percentage of cells which responded to each tastant: DB, denatonium benzoate 10 mM (n=160 cells), 1 mM (n=98 cells), 0.1 mM (n=82 cells); PTC, phenylathiocarbamide 3 mM (n=60 cells); 6-PTU, 6-n-propyl thiouracil 1 mM (n=63 cells); CAP, caffeine 10 mM (n=52); NIC, nicotine 10 mM (n=53 cells); CHL, chloroquine 1 mM (n=52 cells); CYC; cycloheximide 50 μM (n=538 cells). The values in parenthesis are the number of cells analyzed. Lower panels. Individual traces of [Ca²⁺], from two cells exposed to denatonium benzoate or cycloheximide.

FIG. 10 demostrates the effect of denatonium to induce a dose-dependent increase in [Ca²⁺]ᵢ in STC-1 cells. (A) STC-1 cells, grown on cover slips, were washed with a buffer solution (buffer A) consisting of Hanks' balanced salt solution supplemented with 0.03% NaHCO₃, 1.3 mM CaCl₂, 0.5 mM MgCl₂, 0.4 mM MgSO₄, and 0.1% BSA. After washing, cells were incubated with 5 mM fura-2-tetra-acetoxy methyl ester (fura-2/AME) from a stock of 1 mM in DMSO for 30 min at room temperature. Cells were then washed again with buffer A and left at room temperature for an additional 30 min. Each cover slip was placed in a glass cuvette with 2 ml of buffer A, and fluorescence was measured continuously in a Hitachi F-2000 fluorospectrophotometer with excitation wavelength of 340 and 380 nm and an emission wavelength of 510 nm. The bars represent the increase in [Ca²⁺]ᵢ in response to various concentrations of denatonium benzoate (DB), as indicated. (B) [Ca²⁺]ᵢ changes in IEC-18 cells, a normal rat intestinal epithelial cell line, after sequential addition of 5 mM denatonium benzoate (DB) followed by 50 nM vasopressin (VP), added as a positive control. (C) [Ca²⁺]ᵢ changes in Swiss 3T3 cells after sequential addition of 5 mM denatonium benzoate (DB) followed by 10 nM bombesin (Bom), added as a positive control. The changes in [Ca²⁺]ᵢ in IEC-18 and Swiss 3T3 cells were measured as described above for STC-1 cells.

FIG. 11 demonstrates T2R gene organization on mouse chromosome 6. Bitter locus spans about 1.4 Mb and harbors at least 30 T2R-related sequences including 7 pseudogenes. GT2R genes identified from the STC-1 are distributed in two clusters on band 6F3.

### DETAILED DESCRIPTION OF THE INVENTION

The present invention provides novel nucleic acid sequences encoding a family of taste-transducing G-protein coupled receptors from the gastrointestinal tract of mouse, rat and human. This invention also provides for the first time, the identity of known T2R homologs present in the GI tract outside the tongue, and the full sequence of some partially characterized T2R fragments. These nucleic acids and the receptors that they encode are referred to as “GT2R” for gastrointestinal taste receptor, and are designated as GT2R-s (SEQ ID NOS:1-14 from the STC-1 cells), GT2R-m (SEQ ID NOS:15-30 from the mouse GI mucosa), GT2R-r (SEQ ID NOS:31-68 from the rat GI mucosa), and GT2R-h (SEQ ID NOS:69-90 from the human GI cDNA).

These specific GT2Rs and their primary coupling G proteins Gαₜ and Gα_{gust}, are components of the taste transduction pathway (see Example 1). These nucleic acids provide valuable probes for the identification of taste sensing cells, as the nucleic acids are specifically expressed in the GI tract or in a cell line. For example, probes for GT2R polypeptides and proteins can be used to identity subsets of chemosensory cells such as enteric neurons, or specific endocrine cells, e.g. enterochromaffin (EC) cells, enterochromaffin-like (EC) and CCK-producing I cells. Furthermore, the nucleic acids and the proteins they encode can be used as probes to dissect taste-induced behaviors.

The invention also provides methods of screening for ligands/modulators, e.g., activators, inhibitors, stimulators, enhancers, agonists, and antagonists, of these novel GT2Rs. Such modulators of taste transduction are useful for pharmacological and genetic modulation of taste signaling pathways. These methods of screening can be used to identify high affinity agonists and antagonists of GI chemosensory cell activity. These modulatory compounds can then be used in the food and pharmaceutical industries to customize taste sensing in the gut. Thus, the invention provides assays for taste modulation, where GT2R acts as a direct or indirect reporter molecule for the effect of modulators on signal transduction. GT2Rs can be used in assays, e.g., to measure changes in ion concentration, membrane potential, current flow, ion flux, transcription, signal transduction, receptor-ligand interactions, second messenger concentrations, in vitro, in vivo, and ex vivo. In one embodiment, GT2R can be used as an indirect reporter via attachment to a second reporter molecule such as green fluorescent protein (see, e.g., Mistili & Spector, Nature Biotechnology 15:961-964 (1997)). In another embodiment, GT2Rs are expressed in cells, and modulation of signal transduction via GT2R activity is assayed by measuring changes in Ca²⁺ levels (see Example 2).

Methods of assaying for modulators of taste transduction include in vitro ligand binding assays using GT2R-S1, portions thereof such as transmembrane domains, or chimeric proteins comprising one or more domains of GT2R-S1; tissue culture cell GT2R-S1 expression; transcriptional activation of GT2R-S1; phosphorylation and dephosphorylation of GT2Rs; G-protein binding to GT2Rs; ligand binding assays; voltage, membrane potential and conductance changes; ion flux assays; changes in intracellular second messengers such as cAMP and inositol triphosphate; changes in intracellular calcium levels; and hormone or neurotransmitter release.

Finally, the invention provides for methods of detecting GT2R-S1 nucleic acid and protein expression, allowing investigation of taste transduction regulation and specific identification of taste receptor cells. GT2R-Sl is useful as a nucleic acid probe for identifying subpopulations of chemosensory endocrine cells such as EC, ECL, and/or CCK-producing I cells. GT2R-S1 receptors can also be used to generate polyclonal and monoclonal antibodies useful for identifying taste-sensing endocrine cells. These cells can also be identified using techniques such as reverse transcription and amplification of mRNA, isolation of total RNA or poly A⁺ RNA, northern blotting, in situ hybridization, RNase protection, probing DNA microchip arrays, western blots, and the like.

The GT2R genes are part of a large family of bitter taste receptors T2R. In mammalian genome, most T2R genes are present in one of several gene clusters. For example, on mouse chromosome 6, three gene clusters containing 7, 6 and 27 genes (and pseudogenes) have been identified. In human, two gene clusters found on chromosome 12 comprise 14 and 5 genes, while another cluster located on chromosome 7 contains 10 genes. It is estimated that the mouse and human genome each may contain at least 40-50 distinct T2R genes. Chromosomal localization of the genes encoding GT2R can be used to identify diseases, metabolic disorder, and traits associated with GT2R in human, and to develop animal model for dietary supplement and gene targeting studies, which will provide better prevention and therapy to the affected individuals.

Functionally, GT2R represents a seven transmembrane G-protein coupled receptor involved in bitter taste transduction, which interacts with a G-protein (Gαₜ or Gα_{gust} to mediate taste signaling (see, e.g., Fong, Cell Signal 8:217 (1996); Baldwin, Curr. Opin. Cell Biol. 6:180 (1994) and Example 3).

Structurally, the nucleotide sequence of GT2R (see, e.g., SEQ ID NOS:1, 3, 5, or 7 from mouse) encodes a polypeptide of approximately 300-350 amino acids with a predicted molecular weight of approximately 38 kDa and a predicted range of 3540 kDa (see, e.g., SEQ ID NOS:2). GT2R genes from the same species share at least about 50% amino acid identity over a region of at least about 25 amino acids in length, optionally 50 to 100 amino acids in length.

GT2R members are differentially expressed in the GI tract. Similar to mT2R19, GT2R-S1 is abundantly expressed in the antrum, fundus and duodenum, while GT2R-S7 is a moderately abundant sequence found in the same tissues. On the other hand, GT2R-S2 is much less abundant and mT2R21 is hardly expressed in the STC-1 and gastric mucosa (see Example 1 and 4). In addtion to providing nucleic acid probes and primers, the present invention also provides nucleotide sequences for GT2R promoter, which can be used to monitor GI-specific expression of T2R transcription in Gαₜ₋₂ or Gα_{gust} expressing cells in the GI tract and in the STC-1 cell line.

It has been reported that a 4-a.a. difference in mT2R5 resulted in cycloheximide taster (DBA/2J) versus non-taster (C57BL/6J) (Chandrashekar et. al., Cell 100:703-711 (2000)). The present invention also provides polymorphic variants of the GT2R proteins provided herein. For example, GT2R-S2 is highly homologous to mT2R23 with 11-a.a. substitutions, and GT2R-S7 is identical to mT2R2 with only 2-a.a. changes. Partial sequences GT2R-S5-1 are closely related to mT2R7 with 5-a.a. substitutions. The identification of key residue(s) in natural variants or mutants that may enhance or abrogate taste signal transduction thus provide useful target to modify taste molecules.

The identification of GT2R-expressing STC-1 cell system also provides a means for screening for inhibitors and activators of T2R as taste transducer, especially bitter tastants, using in vitro assays to measure ligand binding, G-protein coupling and activation, phosphorylation and dephosphorylation, intracellular 2^{nd} messengers, hormone release (see Example 5). Such activators and inhibitors are useful pharmaceutical and food agents for modifying taste and adding nutrition value.

### II. Definitions

As used herein, the following terms have the meanings ascribed to them unless specified otherwise.

“Gastrointestinal endocrine cells” are hormone and neurotransmitter producing cells that are located in gastric and intestinal glands, e.g., enterochromaffin (EC) cells, enterochromaffin-like (ECL) cells, and cholecystokinin-producing I cells or tumor cell STC-1.

“GT2R” stands for gastrointestinal taste-sensing receptor, refers to a G-protein coupled receptor that is specifically expressed in STC-1 and GI tissues. Such chemosensory cells can be identified because they express specific molecules such as Gα_{gust}, a taste cell specific G protein (McLaughin et al., Nature 357:563-569 (1992)). Endocrine cells can also be identified on the basis of morphology (Norlen et al, J Histochem Cytochem. 49:9-18 (2001)).

GT2R encodes GPCRs with seven transmembrane regions that have “G-protein coupled receptor activity,” e.g., they bind to G-proteins in response to extracellular stimuli and promote production of second messengers such as inositol triphosphate (IP3), cAMP, and Ca²⁺ via stimulation of enzymes such as phospholipase C (PLC) and adenylyl cyclase (for a description of the structure and function of GPCRs, see, e.g., Fong, supra, and Baldwin, supra).

The term GT2R therefore refers to polymorphic variants, alleles, mutants, and interspecies homologs that: (1) have about 60% amino acid sequence identity, to SEQ ID NO:2; SEQ ID NO:4; SEQ ID NO:6; SEQ ID NO:8 over a window of about 25 amino acids, optionally 50-100 amino acids; (2) specifically hybridize (with a size of at least about 500, optionally at least about 900 nucleotides) under stringent hybridization conditions to a sequence selected from the group consisting of SEQ ID NO:1; SEQ ID NO:3; SEQ ID NO:5; SEQ ID NO:7, and conservatively modified variants thereof; or (3) are amplified by primers that specifically hybridize under stringent hybridization conditions to the same sequence as a degenerate primer sets encoding SEQ ID NO:1; SEQ ID NO:3; SEQ ID NO:5; SEQ ID NO:7.

Topologically, taste-sensing GPCRs have a short N-terminal “extracellular domain,” a “transmembrane domain” comprising seven transmembrane regions and corresponding cytoplasmic and extracellular loops, and a C-terminal “cytoplasmic domain” (see, Buck & Axel, Cell 65:175-187 (1991)). These domains can be structurally identified using methods known to those of skill in the art, such as sequence analysis programs that identify hydrophobic and hydrophilic domains (see, e.g., Kyte & Doolittle, J. Mol. Biol. 157:105-132 (1982)). Such domains are useful for making chimeric proteins and for in vitro assays of the invention.

“Extracellular domain” refers to the domains of GT2R polypeptides that protrude from the cellular membrane and are exposed to the external surface of the cell. Such domains would include “N-terminal domain”, and “extracellular loops” between the transmembrane domains (e.g., transmembrane regions 2 and 3, and transmembrane regions 4 and 5).

“Transmembrane domain,” comprising seven transmembrane regions, refers to the domain of GT2R polypeptides that lies within the plasma membrane.

“Cytoplasmic domain” refers to the domain of GT2R polypeptides that face the inside of the cell. Such domins would include “C-terminal domain”, and “intracellular loops” between the transmembrane domains (e.g., transmembrane regions 1 and 2, transmembrane regions 3 and 4). “C-terminal domain” refers to the region that spans the end of the last transmembrane domain and the C-terminus of the polypeptides.

“GPCR activity” refers to the ability of a GPCR to transduce a taste signal. Such activity can be measured in a native cell line (e.g., STC-1) that expressing GPCR, and in heterologous cell, by coupling a GPCR to either a G-protein, G_{gust} or promiscuous G-protein such as Gα₁₅, and an enzyme such as PLC, and measuring increases in intracellular calcium using (Offermans & Simon, J. Biol. Chem. 270:15175-15180 (1995)). Receptor activity can be effectively measured by recording ligand-induced changes in [Ca²⁺]ᵢ using fluorescent Ca²⁺-indicator dyes and fluorometric imaging.

The phrase “functional effects” in the context of assays for testing compounds that modulate GT2R mediated signal transduction includes the determination of any parameter that is indirectly or directly under the influence of the receptor, e.g., functional, physical and chemical effqcts. It includes ligand binding, changes in ion flux, membrane potential, current flow, transcription, G-protein binding, receptor phosphorylation or dephosphorylation, receptor internalization and redistribution, receptor-ligand interactions, second messenger concentrations (e.g., cAMP, IP3, or intracellular Ca²⁺), in vitro, in vivo, and ex vivo and also includes other physiologic effects such increases or decreases of neurotransmitter or hormone release.

By “determining the functional effect” is meant assays for a compound that increases or decreases a parameter that is indirectly or directly under the influence of GT2R, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties, patch clamping, voltage-sensitive dyes, whole cell currents, radioisotope efflux, inducible markers, tissue culture cell GT2R expression; transcriptional activation of GT2R; ligand binding assays; voltage, membrane potential and conductance changes; ion flux assays; changes in intracellular second messengers such as cAMP and IP3; changes in intracellular calcium levels; hormone and neurotransmitter release, and the like.

“Inhibitors,” “activators,” and “modulators” of GT2R are used interchangeably to refer to inhibitory, activating, or modulating molecules identified using in vitro and in vivo assays for signal transduction, e.g., ligands, agonists, antagonists, and their homologs and mimetics. Inhibitors are compounds that, e.g., bind to, partially or totally block stimulation, decrease, prevent, delay activation, inactivate, desensitize, or down regulate taste transduction, e.g., antagonists. Activators are compounds that, e.g., bind to, stimulate, increase, open, activate, facilitate, enhance activation, sensitize or up regulate taste transduction, e.g., agonists. Modulators include compounds that, e.g., alter the interaction of a receptor with: extracellular proteins that bind activators or inhibitor; G-proteins; kinases (e.g., homologs of rhodopsin kinase and beta adrenergic receptor kinases that are involved in deactivation and desensitization of a receptor); and arrestin-like proteins, which also deactivate and desensitize receptors. Modulators include genetically modified versions of GT2R, e.g., with altered activity, as well as naturally occurring and synthetic ligands, antagonists, agonists, small chemical molecules and the like. Such assays for inhibitors and activators include, e.g., expressing GT2R in cells or cell membranes, applying putative modulator compounds, and then determining the functional effects on taste transduction, as described above. Samples or assays comprising GT2R that are treated with a potential activator, inhibitor, or modulator are compared to control samples without the inhibitor, activator, or modulator to examine the extent of inhibition. Control samples (untreated with inhibitors) are assigned a relative GT2R activity value of 100%. Significant inhibition of GT2R is achieved when the GT2R activity value relative to the control is about or 50% or less. Significant activation of GT2R is achieved when the GT2R activity value relative to the control is 150%, optionally 200-500%, or higher.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. “Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are optionally directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed physically linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., 70% identity, optionally 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Optionally, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

One example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403410 (1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

Amplification techniques using primers can also be used to amplify and isolate GT2R from DNA or RNA. The degenerate primers encoding the following amino acid sequences can also be used to amplify a sequence of GT2R from the group consisting of SEQ ID NOS:1, 3, 5 or 7) (see, e.g., Dieffenfach & Dveksler, PCR Primer: A Laboratory Manual (1995)). These primers can be used, e.g., to amplify either the full-length sequence or a probe of one to several hundred nucleotides, which is then used to screen a mammalian library for full-length GT2R-S1.

An alternative method of isolating GT2R nucleic acid homologs combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of GCR-S1 directly from mRNA, from cDNA, from genomic libraries or cDNA libraries. Degenerate oligonucleotides can be designed to amplify GCR-S1 homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of GT2R encoding mRNA in physiological samples, for nucleic acid sequencing, or for other purposes. Genes amplified by the PCR reaction can be purified from agarose gels and cloned into an appropriate vector.

Gene expression of GT2R can also be analyzed by techniques known in the art, e.g., reverse transcription and amplification of mRNA, isolation of total RNA or poly A⁺ RNA, northern blotting, dot blotting, in situ hybridization, RNase protection, probing DNA microchip arrays, and the like. In one embodiment, high density oligonucleotide analysis technology (e.g., GeneChip™, Affymetrix) is used to identify homologs and polymorphic variants of the GT2Rs of the invention. In the case where the homologs being identified are linked to a known disease, they can be used with GeneChip™. as a diagnostic tool in detecting the disease in a biological sample, see, e.g., Gunthand et al., AIDS Res. Hum. Retroviruses 14:869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435448 (1998); Hacia et. al. , Nucleic Acids Res. 26:3865-3866 (1998).

Synthetic oligonucleotides can be used to construct recombinant GT2R genes (e.g., SEQ ID NOS:1, 3, 5, or 7) for use as probes or for expression of protein. This method is performed using a series of overlapping oligonucleotides usually 40-120 bp in length, representing both the sense and nonsense strands of the gene. These DNA fragments are then annealed, ligated and cloned. Alternatively, amplification techniques can be used with precise primers to amplify a specific subsequence of the GT2R nucleic acid (e.g., SEQ ID NOS:1, 3, 5, or 7). The specific subsequence is then ligated into an expression vector.

The nucleic acid encoding GT2R is typically cloned into intermediate vectors before transformation into prokaryotic or eukaryotic cells for replication and/or expression. These intermediate vectors are typically prokaryote vectors, e.g., plasmids, or shuttle vectors.

Alternatively, nucleic acids encoding chimeric proteins comprising GT2R or domains thereof can be made according to standard techniques. For example, a domain such as ligand binding domain, an extracellular domain, a transmembrane domain (e.g., one comprising seven transmembrane regions and corresponding extracellular and cytosolic loops), the transmembrane domain and a cytoplasmic domain, an active site, a subunit association region, etc., can be covalently linked to a heterologous protein. For example, an extracellular domain can be linked to a heterologous GPCR transmembrane domain, or a heterologous GPCR extracellular domain can be linked to a transmembrane domain. Other heterologous proteins of choice include, e.g., green fluorescent protein, β-galactosidase, calcium sensing receptor, and the rhodopsin presequence.

Sequences of interest include those provided in the sequence listing, as set forth in Table 1. The following sequences were determined to be expressed in either gastrointestinal tissues, or cell line, or cDNA libraries from mouse, rat and human. They shared sequence similarity to known T2R that function in taste signal transduction.

TABLE 1

| | | Amino Acid or | | |
|---|---|---|---|---|
| | | Nucleic Acid | | GenBank Acc |
| SEQ ID | Internal code | sequence | other names | No. |
| SEQ ID NO: 1 | GT2R-S1 | n.a. | STC 9-1 (mT2R10) | AF412304 |
| SEQ ID NO: 2 | GT2R-S1 | a.a. | STC 9-1 | AAL85201 |
| SEQ ID NO: 3 | GT2R-S2 | n.a. | STC 9-2, mT2R23 | AF412305 |
| SEQ ID NO: 4 | GT2R-S2 | a.a. | STC 9-2 | AAL85202 |
| SEQ ID NO: 5 | GT2R-S7 | n.a. | STC 9-7, mT2R2 | AF412306 |
| SEQ ID NO: 6 | GT2R-S7 | a.a. | STC 9-7 | AAL85203 |
| SEQ ID NO: 7 | GT2R-S8 | n.a. | STC 9-8 | (NOVEL) |
| SEQ ID NO: 8 | GT2R-S8 | a.a. | STC 9-8 | |
| SEQ ID NO: 9 | GT2R-S5-1 | n.a. | STC 5-1, mT2R7 | AF412301 |
| SEQ ID NO: 10 | GT2R-S5-1 | a.a. | STC 5-1 | AAL85198.1 |
| SEQ ID NO: 11 | GT2R-S7-1 | n.a | STC 7-1 | AF412302 |
| SEQ ID NO: 12 | GT2R-S7-1 | a.a. | STC 7-1 | AAL85199.1 |
| SEQ ID NO: 13 | GT2R-S7-4 | n.a. | STC 7-4 | AF412303 |
| SEQ ID NO: 14 | GT2R-S7-4 | a.a. | STC 7-4 | AAL85200 |
| SEQ ID NO: 15 | GT2R-m33 | n.a. | 619A | (NOVEL) |
| SEQ ID NO: 16 | GT2R-m33 | a.a. | | |
| SEQ ID NO: 17 | GT2R-m34 | n.a | 088 | (NOVEL) |
| SEQ ID NO: 18 | GT2R-m34 | a.a. | | |
| SEQ ID NO: 19 | GT2R-m35 | n.a. | 273A | (NOVEL) |
| SEQ ID NO: 20 | GT2R-m35 | a.a. | | |
| SEQ ID NO: 21 | GT2R-m36 | n.a. | 273B | (NOVEL) |
| SEQ ID NO: 22 | GT2R-m36 | a.a | | |
| SEQ ID NO: 23 | GT2R-m37 | n.a. | 273C | (NOVEL) |
| SEQ ID NO: 24 | GT2R-m37 | a.a. | | |
| SEQ ID NO: 25 | GT2R-m38 | n.a. | 273D | (NOVEL) |
| SEQ ID NO: 26 | GT2R-m38 | a.a. | | |
| SEQ ID NO: 27 | GT2R-m39 | n.a. | 625A | (NOVEL) |
| SEQ ID NO: 28 | GT2R-m39 | a.a. | | |
| SEQ ID NO: 29 | GT2R-m41 | n.a. | 923 | (NOVEL) |
| SEQ ID NO: 30 | GT2R-m41 | a.a. | | |
| SEQ ID NO: 31 | GT2R-r6 | n.a | rT2R6 | AF240766 |
| | | | | (partial) |
| SEQ ID NO: 32 | GT2R-r6 | a.a | rT2R6 full-length | |
| SEQ ID NO: 33 | GT2R-r14 | n.a. | rT2R14 full-length | |
| SEQ ID NO: 34 | GT2R-r14 | a.a. | rT2R14 full-length | |
| SEQ ID NO: 35 | GT2R-r15 | n.a. | 912A | |
| SEQ ID NO: 36 | GT2R-r15 | a.a | | |
| SEQ ID NO: 37 | GT2R-r16 | n.a. | | |
| SEQ ID NO: 38 | GT2R-r16 | a.a | | |
| SEQ ID NO: 39 | GT2R-r17 | n.a. | | |
| SEQ ID NO: 40 | GT2R-r17 | a.a. | | |
| SEQ ID NO: 41 | GT2R-r18 | n.a. | | |
| SEQ ID NO: 42 | GT2R-r18 | a.a | | |
| SEQ ID NO: 43 | GT2R-r19 | n.a. | 094B | |
| SEQ ID NO: 44 | GT2R-r19 | a.a. | | |
| SEQ ID NO: 45 | GT2R-r20 | n.a. | | |
| SEQ ID NO: 46 | GT2R-r20 | a.a. | | |
| SEQ ID NO: 47 | GT2R-r21 | n.a. | rD4081 | |
| SEQ ID NO: 48 | GT2R-r21 | a.a. | | |
| SEQ ID NO: 49 | GT2R-r22 | n.a. | rD4082 | |
| SEQ ID NO: 50 | GT2R-r22 | a.a. | | |
| SEQ ID NO: 51 | GT2R-r23 | n.a. | 503A | |
| SEQ ID NO: 52 | GT2R-r23 | a.a. | | |
| SEQ ID NO: 53 | GT2R-r24 | n.a. | 503B | |
| SEQ ID NO: 54 | GT2R-r24 | a.a. | | |
| SEQ ID NO: 55 | GT2R-r25 | n.a. | | |
| SEQ ID NO: 56 | GT2R-r25 | a.a. | | |
| SEQ ID NO: 57 | GT2R-r26 | n.a. | | |
| SEQ ID NO: 58 | GT2R-r26 | a.a. | | |
| SEQ ID NO: 59 | GT2R-r27 | n.a. | | |
| SEQ ID NO: 60 | GT2R-r27 | a.a. | | |
| SEQ ID NO: 61 | GT2R-r28 | n.a | | |
| SEQ ID NO: 62 | GT2R-r28 | a.a. | | |
| SEQ ID NO: 63 | GT2R-r29 | n.a. | | |
| SEQ ID NO: 64 | GT2R-r29 | a.a. | | |
| SEQ ID NO: 65 | GT2R-r30 | n.a. | | |
| SEQ ID NO: 66 | GT2R-r30 | a.a. | | |
| SEQ ID NO: 67 | GT2R-r31 | n.a | rD4232 | |
| SEQ ID NO: 68 | GT2R-r31 | a.a | | |
| SEQ ID NO: 69 | GT2R-h44 | n.a. | 630G, hT2R44 | |
| SEQ ID NO: 70 | GT2R-h44 | a.a | | |
| SEQ ID NO: 71 | GT2R-h50 | n.a. | | |
| SEQ ID NO: 72 | GT2R-h50 | a.a | gastric peptide ZG24 | |
| SEQ ID NO: 73 | GT2R-h51 | n.a | 630C | |
| SEQ ID NO: 74 | GT2R-h51 | a.a | | |
| SEQ ID NO: 75 | GT2R-h52 | n.a. | 630D | |
| SEQ ID NO: 76 | GT2R-h52 | a.a. | | |
| SEQ ID NO: 77 | GT2R-h53 | n.a. | 630E | |
| SEQ ID NO: 78 | GT2R-h53 | a.a. | | |
| SEQ ID NO: 79 | GT2R-h54 | n.a. | 630H | |
| SEQ ID NO: 80 | GT2R-h54 | a.a | | |
| SEQ ID NO: 81 | GT2R-h55 | n.a. | 630F | |
| SEQ ID NO: 82 | GT2R-h55 | a.a | | |
| SEQ ID NO: 83 | GT2R-h56 | n.a. | 630I | |
| SEQ ID NO: 84 | GT2R-h56 | a.a. | | |
| SEQ ID NO: 85 | GT2R-h57 | n.a. | 630J | |
| SEQ ID NO: 86 | GT2R-h57 | a.a | | |
| SEQ ID NO: 87 | GT2R-h58 | n.a. | 640A | |
| SEQ ID NO: 88 | GT2R-h58 | a.a. | | |
| SEQ ID NO: 89 | GT2R-h59 | n.a. | hT2R38 | |
| SEQ ID NO: 90 | GT2R-h59 | a.a. | | |

### Assays for GT2R Activity

GT2R-Sl and its alleles, polymorphic variants and homologs are G-protein coupled receptors that participate in taste transduction. The activity of GT2R polypeptides can be assessed using a variety of in vitro and in vivo assays to determine functional, chemical, and physical effects, e.g., measuring ligand binding (e.g., radioactive ligand binding), second messengers (e.g., cAMP, cGMP, IP₃, DAG, or Ca²⁺), ion flux, phosphorylation levels, transcription levels, neurotransmitter levels, and the like. Furthermore, such assays can be used to test for inhibitors and activators of GT2R. Modulators can also be genetically altered versions of GT2R. Such modulators of taste transduction activity are useful for customizing taste.

The GT2R of the assay will be selected from a polypeptide having a sequence selected from a group consisting of SEQ ID NOS:2, 4, 6, 8, or conservatively modified variant thereof. Alternatively, the GT2R of the assay will be derived from a eukaryote and include an amino acid subsequence having amino acid sequence identity SEQ ID NOS:2, 4, 6, or 8. Generally, the amino acid sequence identity will be at least 70%, optionally at least 85%, optionally at least 90-95%. Optionally, the polypeptide of the assays will comprise a domain of the selected GT2R, such as an extracellular domain, transmembrane domain, cytoplasmic domain, ligand binding domain, subunit association domain, active site, and the like. Either the whole GT2R polypeptide or a domain thereof can be covalently linked to a heterologous protein to create a chimeric protein used in the assays described herein.

Modulators of GT2R-S1 activity are tested using selected GT2R polypeptides as described above, either recombinant or naturally occurring. The protein can be isolated, expressed in a cell, expressed in a membrane derived from a cell, expressed in tissue or in an animal, either recombinant or naturally occurring. For example, gastric gland, dissociated cells from GI mucosa, transformed cells (e.g. STC-1), or membranes can be used. Modulation is tested using one of the in vitro or in vivo assays described herein. Taste transduction can also be examined in vitro with soluble or solid state reactions, using a chimeric molecule such as an extracellular domain of a receptor covalently linked to a heterologous signal transduction domain, or a heterologous extracellular domain covalently linked to the transmembrane and or cytoplasmic domain of a receptor. Furthermore, ligand-binding domains of the protein of interest can be used in vitro in soluble or solid state reactions to assay for ligand binding.

Ligand binding to GT2R whole protein, a domain, or chimeric protein can be tested in solution, in a bilayer membrane, attached to a solid phase, in a lipid monolayer, or in vesicles. Binding of a modulator can be tested using, e.g., changes in spectroscopic characteristics (e.g., fluorescence, absorbance, refractive index) hydrodynamic (e.g., shape), chromatographic, or solubility properties.

Receptor-G-protein interactions can also be examined. For example, binding of the G-protein to the receptor or its release from the receptor can be examined. For example, in the absence of GTP, an activator will lead to the formation of a tight complex of a G protein (all three subunits) with the receptor. This complex can be detected in a variety of ways, as noted above. Such an assay can be modified to search for inhibitors. Add an activator to the receptor and G protein in the absence of GTP, form a tight complex, and then screen for inhibitors by looking at dissociation of the receptor-G protein complex. In the presence of GTP, release of the alpha subunit of the G protein from the other two G protein subunits serves as a criterion of activation.

An activated or inhibited G-protein will in turn alter the properties of target enzymes, channels, and other effector proteins. The classic examples are the activation of cGMP phosphodiesterase by transducin in the visual system, adenylyl cyclase by the stimulatory G-protein, phospholipase C by Gq and other cognate G proteins, and modulation of diverse channels by Gi and other G proteins. Downstream consequences can also be examined such as generation of diacyl glycerol and IP3 by PLC, and in turn, for calcium mobilization by IP3.

Activated GPCR receptors become substrates for kinases that phosphorylate the C-terminal tail of the receptor (and possibly other sites as well). Thus, activators will promote the transfer of 32p from gamma-labeled GTP to the receptor, which can be assayed with a scintillation counter. The phosphorylation of the C-terminal tail will promote the binding of arrestin-like proteins and will interfere with the binding of G-proteins. The kinase/arrestin pathway plays a key role in the desensitization of many GPCR receptors. For example, compounds that modulate the duration a taste receptor stays active would be useful as a means of prolonging a desired taste or cutting off an unpleasant one. For a general review of GPCR signal transduction and methods of assaying signal transduction, see, e.g., Methods in Enzymology, vols. 237 and 238 (1994) and volume 96 (1983); Bourne et al., Nature 10:349:117-27 (1991); Bourne et al., Nature 348:125-32 (1990); Pitcheret al., Annu. Rev. Biochem. 67:653-92 (1998).

Samples or assays that are treated with a potential GT2R inhibitor or activator are compared to control samples without the test compound, to examine the extent of modulation. Control samples (untreated with activators or inhibitors) are assigned a relative GT2R activity value of 100. Inhibition of GT2R is considered significant when the GT2R activity value relative to the control is 80%, optionally 50% or lower. Activation of GT2R is achieved when the GT2R activity value relative to the control is 150%, preferably 200-500%, or higher.

The effects of the test compounds upon the function of the polypeptides can be measured by examining any of the parameters described above. Any suitable physiological change that affects GPCR activity can be used to assess the influence of a test compound on the polypeptides of this invention. When the functional consequences are determined using intact cells or animals, one can also measure a variety of effects such as transmitter release, hormone release, transcriptional changes to both known and uncharacterized genetic markers (e.g., northern blots), changes in cell metabolism such as cell growth or pH changes, and changes in intracellular second messengers such as Ca²⁺, 1P3 or cAMP.

Preferred assays for G-protein coupled receptors include cells that are loaded with ion or voltage sensitive dyes to report receptor activity. Assays for determining activity of such receptors can also use known agonists and antagonists for other G-protein coupled receptors as negative or positive controls to assess activity of tested compounds. In assays for identifying modulatory compounds (e.g., agonists, antagonists), changes in the level of ions in the cytoplasm or membrane voltage will be monitored using an ion sensitive or membrane voltage fluorescent indicator, respectively. Among the ion-sensitive indicators and voltage probes that may be employed are those disclosed in the Molecular Probes 1997 Catalog. For G-protein coupled receptors, promiscuous G-proteins such as Gα15 and Gα16 can be used in the assay of choice (Wilkie et al., Proc. Nat'l Acad. Sci. USA 88:10049-10053 (1991)). Such promiscuous G-proteins allow coupling of a wide range of receptors.

Receptor activation typically initiates subsequent intracellular events, e.g., increases in second messengers such as IP3, which releases intracellular stores of calcium ions. Activation of some G-protein coupled receptors stimulates the formation of IP3 through phospholipase C-mediated hydrolysis of phosphatidylinositol (Berridge & Irvine, Nature 312:315-21 (1984)). IP3 in turn stimulates the release of intracellular calcium ion stores. Thus, a change in cytoplasmic calcium ion levels, or a change in second messenger levels such as IP3 can be used to assess G-protein coupled receptor function. Cells expressing such G-protein coupled receptors may exhibit increased cytoplasmic calcium levels as a result of contribution from both intracellular stores and via activation of ion channels, in which case it may be desirable although not necessary to conduct such assays in calcium-free buffer, optionally supplemented with a chelating agent such as EGTA, to distinguish fluorescence response resulting from calcium release from internal stores.

Other assays can involve determining the activity of receptors which, when activated, result in a change in the level of intracellular cyclic nucleotides, e.g., cAMP or cGMP, by activating or inhibiting enzymes such as adenylyl cyclase. There are cyclic nucleotide-gated ion channels, e.g., rod photoreceptor cell channels and olfactory neuron channels that are permeable to cations upon activation by binding of cAMP or cGMP (see, e.g., Altenhofen et al., Proc. Natl. Acad. Sci. U.S.A. 88:9868-9872 (1991) and Dhallan et al., Nature 347:184-187 (1990)). In cases where activation of the receptor results in a decrease in cyclic nucleotide levels, it may be preferable to expose the cells to agents that increase intracellular cyclic nucleotide levels, e.g., forskolin, prior to adding a receptor-activating compound to the cells in the assay. Cells for this type of assay can be made by co-transfection of a host cell with DNA encoding a cyclic nucleotide-crated ion channel, GPCR phosphatase and DNA encoding a receptor (e.g., certain glutamate receptors, muscarinic acetylcholine receptors, dopamine receptors, serotonin receptors, and the like), which, when activated, causes a change in cyclic nucleotide levels in the cytoplasm.

In one embodiment, GT2R activity is measured by expressing a selected GT2R (e.g., GT2R-S1, SEQ ID NO:1) in a heterologous cell with a promiscuous G-protein that links the receptor to a PLCC signal transduction pathway (see Offermanns & Simon, J. Biol. Chem. 270:15175-15180 (1995); see also Example 2). Optionally the cell line is HEK-293 (which does not naturally express GT2R) and the promiscuous G-protein is Gα₁₅ (Offermanns & Simon, supra). Modulation of taste transduction is assayed by measuring changes in intracellular Ca²⁺ levels, which change in response to modulation of the GT2R signal transduction pathway via administration of a molecule that associates with this particular GT2R. Changes in Ca²⁺ levels are optionally measured using fluorescent Ca²⁺ indicator dyes and fluorometric imaging.

In another embodiment, the changes in intracellular cAMP or cGMP can be measured using immunoassays. The method described in Offermanns & Simon, J. Biol. Chem. 270:15175-15180 (1995) may be used to determine the level of CAMP. Also, the method described in Felley-Bosco et al., Am. J. Resp. Cell and Mol. Biol. 11:159-164 (1994) may be used to determine the level of cGMP. Further, an assay kit for measuring cAMP and/or cGMP is described in U.S. Pat. No. 4,115,538, herein incorporated by reference.

In another embodiment, phosphatidyl inositol (PI) hydrolysis can be analyzed according to U.S. Pat. No. 5,436,128, herein incorporated by reference. Briefly, the assay involves labeling of cells with ³H-myoinositol for 48 h. The labeled cells are treated with a test compound for one hour. The treated cells are lysed and extracted in chloroform-methanol-water after which the inositol phosphates were separated by ion exchange chromatography and quantified by scintillation counting. Fold stimulation is determined by calculating the ratio of cpm in the presence of agonist to cpm in the presence of buffer control. Likewise, fold inhibition is determined by calculating the ratio of cpm in the presence of antagonist to cpm in the presence of buffer control (which may or may not contain an agonist).

In another embodiment, transcription levels can be measured to assess the effects of a test compound on signal transduction. A host cell containing the protein of interest is contacted with a test compound for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots or their polypeptide products may be identified using immunoassays. Alternatively, transcription based assays using reporter gene may be used as described in U.S. Pat. No. 5,436,128, herein incorporated by reference. The reporter genes can be, e.g., chloramphenicol acetyltransferase, firefly luciferase, bacterial luciferase, β-galactosidase and alkaline phosphatase. Furthermore, the protein of interest can be used as an indirect reporter via attachment to a second reporter such as green fluorescent protein (see, e.g., Mistili & Spector, Nature Biotechnology 15:961-964 (1997)).

The amount of transcription is then compared to the amount of transcription in either the same cell in the absence of the test compound, or it may be compared with the amount of transcription in a substantially identical cell that lacks the protein of interest. A substantially identical cell may be derived from the same cells from which the recombinant cell was prepared but which had not been modified by introduction of heterologous DNA. Any difference in the amount of transcription indicates that the test compound has in some manner altered the activity of the protein of interest.

### Modulators for GT2R

The compounds tested as modulators of GT2R can be any small chemical compound, or a biological entity, such as a protein, amino acid, sugar, nucleic acid or lipid. Alternatively, modulators can be genetically altered versions of GT2R. Typically, test compounds will be small chemical molecules and peptides. Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microciter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCTIUS96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Wobum, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

### Computer-based Assays

Yet another assay for compounds that modulate GT2R activity involves computer assisted drug design, in which a computer system is used to generate a three-dimensional structure of GT2R based on the structural information encoded by the amino acid sequence. The input amino acid sequence interacts directly and actively with a preestablished algorithm in a computer program to yield secondary, tertiary, and quaternary structural models of the protein. The models of the protein structure are then examined to identify regions of the structure that have the ability to bind, e.g., ligands. These regions are then used to identify ligands that bind to the protein.

The three-dimensional structural model of the protein is generated by entering protein amino acid sequences of at least 10 amino acid residues or corresponding nucleic acid sequences encoding a GT2R polypeptide into the computer system. The amino acid sequence of the polypeptide of the nucleic acid encoding the polypeptide is selected from the group consisting of SEQ ID NOS:2, 4, 6 or 8 and conservatively modified versions thereof. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM, DVD), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of the GT2R-S1 protein to identify ligands that bind to GT2R-S1. Binding affinity between the protein and ligands is determined using energy terms to determine which ligands have an enhanced probability of binding to the protein.

Computer systems are also used to screen for mutations, polymorphic variants, alleles and interspecies homologs of selected GT2R genes. Such mutations can be associated with disease states or genetic traits. As described above, GeneChip™ and related technology can also be used to screen for mutations, polymorphic variants, alleles and interspecies homologs. Once the variants are identified, diagnostic assays can be used to identify patients having such mutated genes. Identification of the mutated GT2R genes involves receiving input of a first nucleic acid or amino acid sequence, selected from the group consisting of SEQ ID NOS:1, 3, 5 or 7, or SEQ ID NOS:2, 4, 6 or 8 and conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in GT2R genes, and mutations associated with disease states and genetic traits.

### Kits

GT2R homologs are useful tools for identifying taste-sensing cells, for forensics and paternity determinations, and for examining taste transduction. GT2R-specific reagents that specifically hybridize to GT2R nucleic acid, such as GT2R-S1 probes and primers, and GT2R-specific reagents that specifically bind to the GT2R proteins, e.g., GT2R antibodies are used to examine gastrointestinal taste cell expression and taste transduction regulation.

Nucleic acid assays for the presence of GT2R DNA and RNA in a sample include numerous techniques are known to those skilled in the art, such as Southern analysis, northern analysis, dot blots, RNase protection, S1 analysis, amplification techniques such as RT-PCR and QPCR, and in situ hybridization. In in situ hybridization, for example, the target nucleic acid is liberated from its cellular surroundings in such as to be available for hybridization within the cell while preserving the cellular morphology for subsequent interpretation and analysis . The following articles provide an overview of the art of in situ hybridization: Singer et al., Biotechniques 4:230-250 (1986); Haase et al., Methods in Virology, vol. VlI, pp. 189-226 (1984); and Nucleic Acid Hybridization: A Practical Approach (Hames et al., eds. 1987).

In addition, GT2R protein can be detected with the various immunoassay techniques described above. The test sample is typically compared to both a positive control (e.g., a sample expressing recombinant GT2R) and a negative control.

The present invention also provides for kits for screening for modulators of a specific GT2R. Such kits can be prepared from readily available materials and reagents. For example, such kits can comprise any one or more of the following materials: GT2R nucleic acids or proteins, reaction tubes, and instructions for testing GT2R activity. Optionally, the kit contains biologically active GT2R. A wide variety of kits and components can be prepared according to the present invention, depending upon the intended user of the kit and the particular needs of the user.

### EXAMPLES

The following examples are provided by way of illustration only and not by way of limitation. Those of skill in the art will readily recognize a variety of noncritical parameters that could be changed or modified to yield essentially similar results.

### Example 1

### Expression of G_{gust} Gₜ₋₂ and GT2R in STC-1 Cells

The enteroendocrine cells play a critical role in the integration and coordination of multiple physiological responses including motility, release of gastrointestinal hormones and pancreatobiliary secretion. We hypothesized that these cells could play a role in sensing the chemical composition of the luminal contents. As a first step towards testing this hypothesis, we examined whether the intestinal STC-1 cell line, a mixed population of gut endocrine cells (Rindi et. al., Amer. J. Pathol. 136:1349-1363 (1990)), express members of the T2R family as well as Gα subunits of G proteins implicated in intracellular taste signal transduction.

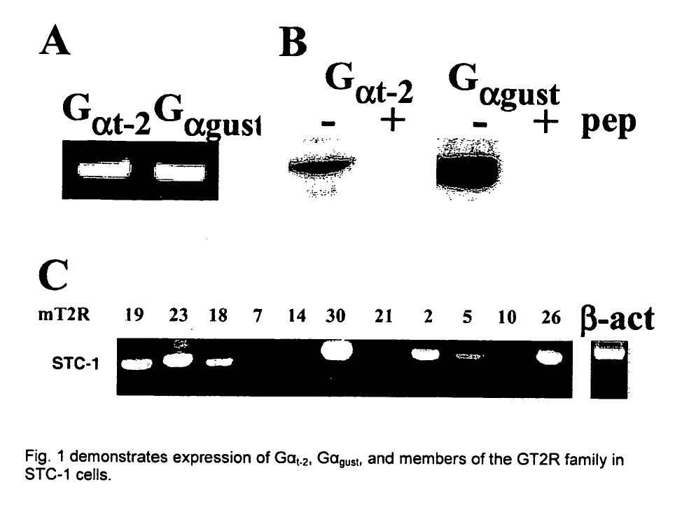

RT-PCR and sequencing revealed the presence of transcripts for Gα_{gust} and Gαₜ₋₂ in STC-1 cells (FIG. 1A). Furthermore, Western blot analysis of STC-1 cell lysates using specific antibodies directed against Gα_{gust} and Gαₜ₋₂ revealed immunoreactive bands of 42-46 kDa which were extinguished by the presence of the immunogenic peptide (FIG. 1 B). These results demonstrate that STC-1 cells express the α subunits of G proteins implicated in intracellular taste signal transduction and thus, prompted us to examine whether these enteroendocrine cells could also express bitter taste receptors.

To test if members of the T2R family are expressed in STC-1 cells, we initially used mouse T2R subtype-specific primers based on the available sequence of mT2R5, mT2R8 and mT2R19. RT-PCR and sequencing analysis, revealed the presence of mT2R5 and mT2Rl9. The taste receptors from the STC-1 cells are identical to those from the taste cells, as shown by sequencing the cDNA encoding the full-length receptor proteins of mT2R19 and mT2R5 from these cells. In contrast, none of these transcripts were detected by RT-PCR using RNA isolated from mouse Swiss 3T3 fibroblasts.

In order to determine whether other members of the bitter taste receptor family are expressed in STC-1 cells, cross-species and degenerate primers were used to amplify mouse STC-1 cell cDNA. RT-PCR and sequencing analysis demonstrated that STC-1 cells expressed mT2R19, mT2R23, mT2R18, mT2R7, mT2R30, mT2R2, mT2R5, and mT2R26 (FIG. 1C) and novel T2R genes.

We used specific primers to amplify the mouse gene fragments from genomic DNA and to screen for these mouse genes in two genomic DNA libraries (BAC mouse ES-129/SvJ rel. I and II, Incyte Genomics, St. Louis, Mo.). Seven genomic clones were obtained and four GT2R genes (corresponding to STC-1 cDNA clones S-1, S-2, S-7 and S-8) were found in these genomic DNA clones.

### Example 2

### GT2R from STC-1 is a Taste-sensing Receptor

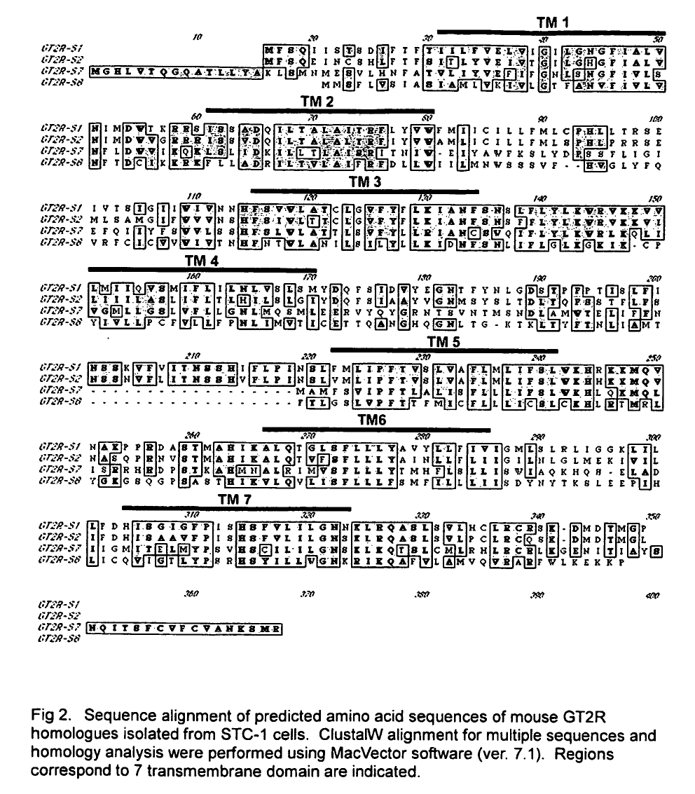

Amino acid sequences were deduced from cDNA clones of STC-1 or from the genomic clones isolated from the mouse genomic DNA libraries (FIG. 2). This analysis revealed that mouse GT2R-S1 and S4 are novel sequences that have 84 and 75% homology to rT2R2 and GT2R-r22, respectively. Mouse GT2R-S1 was further found to express in mouse fundic, antral and duodenal mucosa tissues. The mouse GT2R-S2 gene is closely related to mT2R23 with 7 amino acid substitutions and the GT2R-S7 is almost identical to mT2R2 with only 2 amino acid changes. The latter two transcripts may represent variants of existing mT2R. Similarly, mouse cDNA clones GT2R-S5-1 and S7-4 are virtually identical to mT2R7 and mT2R30 and thus may be the variant forms of these genes. However, genes encoding full-length GT2R proteins are needed to confirm their true identity.

Having demonstrated that STC-1 cells express Gα_{gust}, Gαₜ₋₂ and multiple GT2Rs, we examined whether the addition of bitter taste compounds induces a functional response in these cells. Due to heterogeneity of the STC-1 cell population, we monitored responses in the intracellular Ca²⁺ concentration ([Ca²⁺]ᵢ) using Ca²⁺ imaging of individual cells and tested the effect of several compounds widely used in bitter taste signaling.

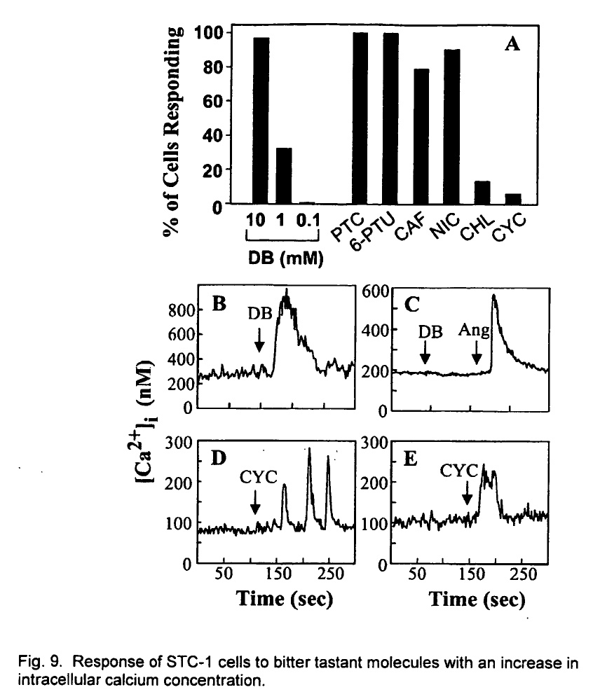

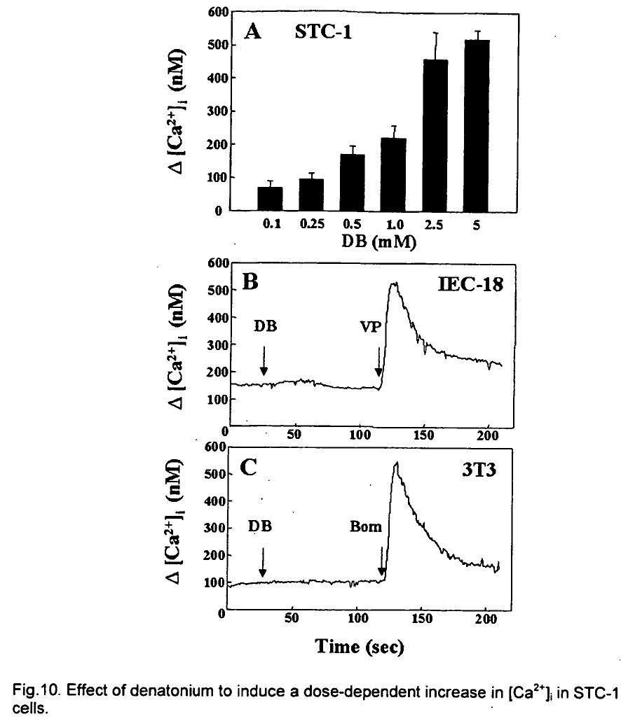

Addition of denatonium benzoate to cultures of STC-1 cells, loaded with the fluorescence Ca²⁺ indicator fura 2-AM, induced a rapid and dose-dependent elevation in the intracellular Ca²⁺ concentration ([Ca²⁺]ᵢ). At 10 mM, denatonium induced a marked increase in [Ca²⁺]i in 97% of the cells examined whereas at 1 mM, this bitter tastant induced an increase in [Ca²⁺]i in 33% of the STC-1 population. The concentrations of denatonium used in these experiments are similar to those used for eliciting second messenger changes and ion channel activity in taste tissues. A variety of other bitter substances including phenylthiocarbamide, 6-n-propil-2-thiouracil, caffeine and nicotine also induced robust [Ca²⁺]i responses in STC-1 cells (FIGS. 9 and 10).

Heterologous expression in HEK-293 cells of chimeric mT2R5 receptors containing the NH2-terminal 39 amino acids of rhodopsin has demonstrated that mT2R-5 responds to cycloheximide, as shown by an increase in [Ca²⁺]ᵢ (Chandrashekar et al, Cell 100:703-711 (2000)). Since a low level of mT2R5 expression was also detected in STC-1 cells, we determined if cycloheximide stimulates a [Ca²⁺]ᵢ response in these cells. We found that addition of cycloheximide induced oscillatory changes in [Ca²⁺]i in a small sub-population of STC-1 cells (FIG. 9). In contrast, other bitter substances including atropine, caffeic acid and epicatechin did not induce any detectable change in [Ca⁺]ᵢ in STC-1 cells.

### Example 3

### Expression of Gustducin (Gα_{gust}) and Transducin (Gₜ) in Rat and Mouse GI Tissues

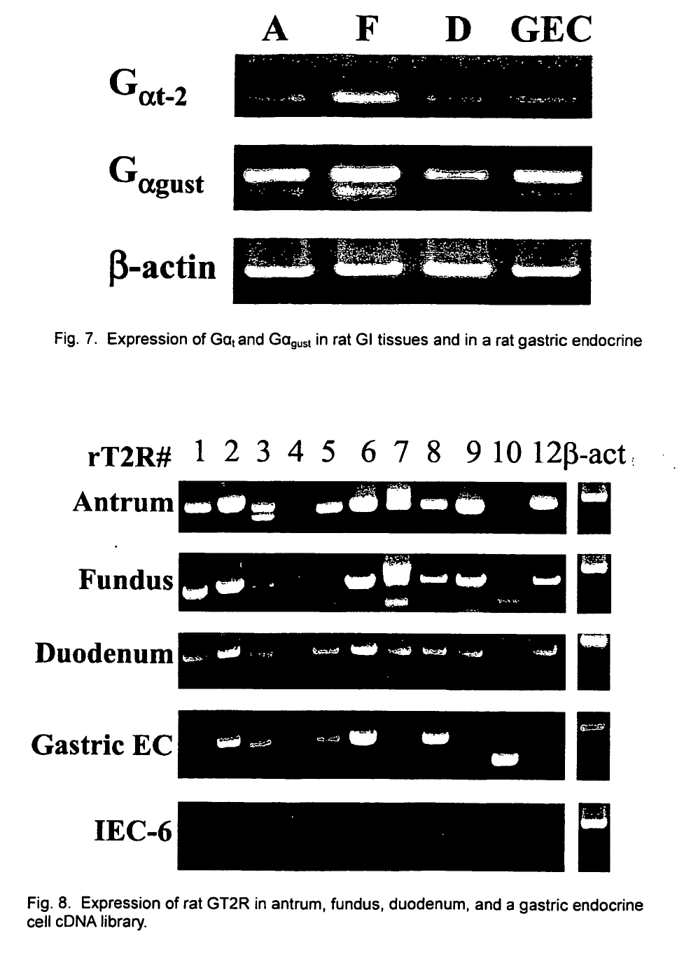

In order to identify Gα_{gust} expression in the GI tract, reversed transcribed mRNA isolated from rat antral, fundic and duodenal mucosa was subjected to PCR using specific primers based on the rat Gα_{gust} sequence (FIG. 7). A major PCR product of the predicted size (332 bp) was obtained from each of these tissues. Interestingly, PCR amplification of a cDNA library enriched in rat gastric endocrine cells using the Gα_{gust} specific primers also produced a 332 bp fragment. Sequence analysis verified that these PCR products corresponded to amplified G_{gust}. We confirmed the identity of gastric Gα_{gust} by cloning and sequencing cDNA fragments encoding the entire open reading frame of Gα_{gust} from the gastric endocrine cell cDNA library.

The α subunit of transducins 1 (Gαₜ₋₁) and 2 (Gαₜ₋₂), originally thought to be expressed only in photoreceptor cells of the retina, are also present in vertebrate taste cells where are implicated in intracellular taste signal transduction. Here, we used RT-PCR and immunohistochemistry to identify that the transducins are also expressed in the GI mucosa.

RT-PCR using primers based on the consensus sequence encoding the COOH-terminal 114 amino acids of human and mouse transducins (described in FIG. 1) detected Gαₜ₋₂ transcripts predominantly in the fundic mucosa. Weaker RT-PCR signals were also obtained with RNA extracted from the gastric antrum and duodenum. In addition, Gαₜ₋₂ was also detected in the cDNA library of rat gastric endocrine cells. In contrast, using the same conditions of RT-PCR, only faint signals corresponding to amplified Gαₜ₋₁ were obtained from fundus, antrum, duodenum and the cDNA library of rat gastric endocrine cells. When RT-PCR for Gαₜ₋₁ was performed for 15 additional cycles, the predicted Gαₜ₋₁ product (340 bp) was detected only in the fundus, while an unspliced variant containing an additional 116 bp intron 7 (456 bp) was identified in the antrum (data not shown). These results indicate that, in addition to Gα_{gust}, the transducins, especially Gαₜ₋₂, are also expressed in the gastrointestinal system.

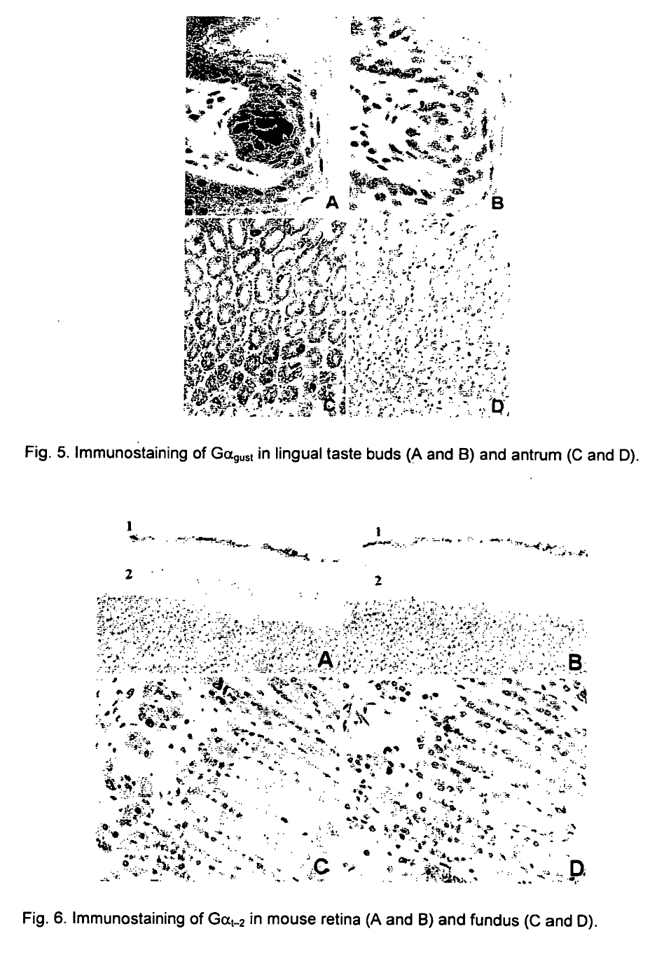

While Gα_{gust} transcripts were detected in the fundus and in the antrum, Gαₜ₋₂ appeared to be present preferentially in the fundus, suggesting that these two Gα, proteins might be expressed by different gastric cells, in order to explore this possibility, we examined the expression of the α subunits of these G proteins by immunohistochemistry using specific antibodies directed against unique amino acid sequences of Gα_{gust} and Gαₜ₋₂. In sections of mouse fundic mucosa, Gαₜ₋₂ was localized to cells present in the base rather than the apical region of the glands. In the neck, only few scattered Gαₜ₋₂ positive cells were seen. Conversely, most Gα_{gust}-positive cells of the fundus were located in the upper (neck) region of the glands, in the isthmus or in the surface epithelium but not in the basal portion. Gαₜ₋₂ staining cells were found rarely in the antral mucosa whilst Gα_{gust}-positive cells were abundant in that zone of the stomach. Exposure of the Gαₜ₋₂ and Gα_{gust} antibodies to the corresponding immunogenic peptides completely abolished immunostaining of the gastric epithelial cells. As revealed by examination of serial sections, the distribution and morphology (see inserts) of Gαₜ₋₂-positive cells were clearly different from those of Gα_{gust}-positive cells (FIG. 4-6). The findings indicate that Gα_{gust} and Gαₜ₋₂ are expressed by distinct epithelial cell types in the gastric mucosa.

### Example 4

Identification of putative GT2R transcripts in the rat and mouse GI mucosa Since gustducin and transducins, which are implicated in bitter taste receptor signal transduction, are expressed in the GI tract, the next step was to determine whether any member of the taste receptor families identified in taste cells of the lingual epithelium are also expressed in the gastric and duodenal mucosa. No transcripts of the T1R families in the mouse or rat gastric or duodenal mucosa were detected. In striking contrast, RT-PCR analysis using degenerate T2R-primers produced amplification products in the antrum, fundus and duodenum.

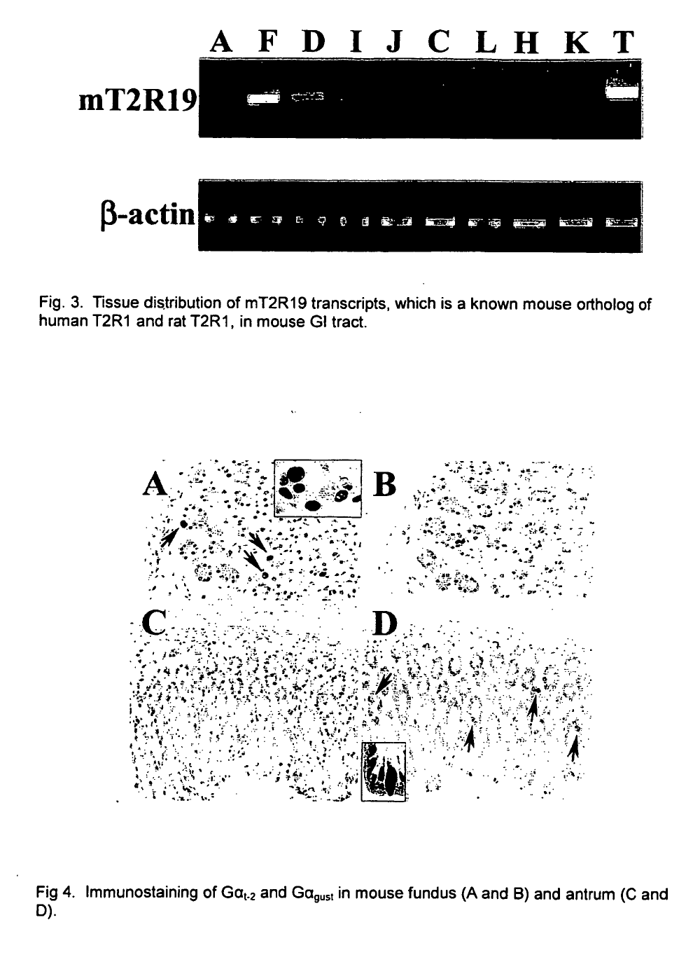

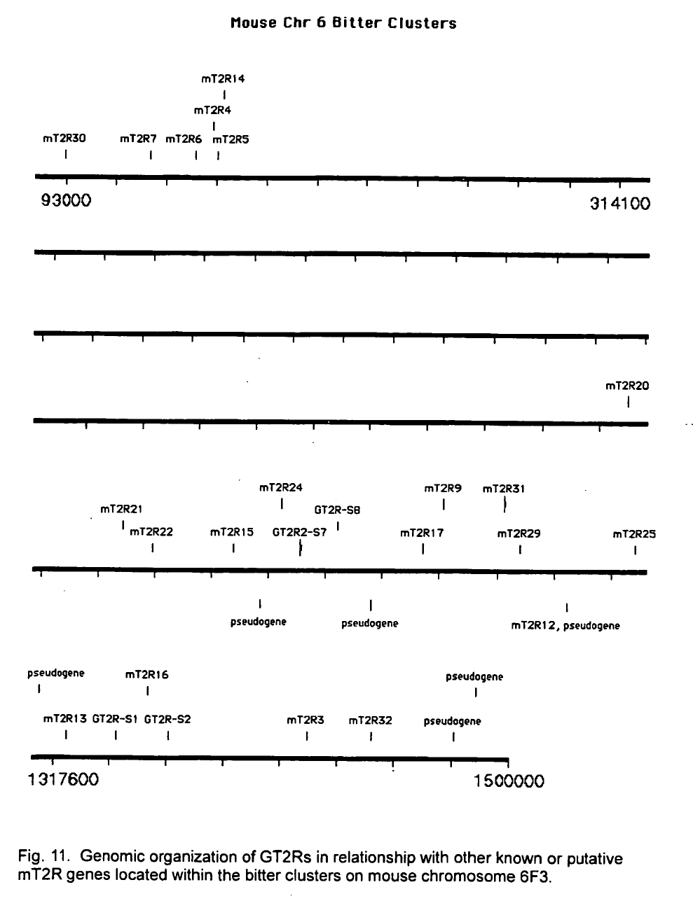

We further examined the expression of known T2R subtypes in the rat antral, fundic and duodenal tissues. RT-PCR using rat-specific primers detected multiple T2R transcripts in rat antrum, fundus and duodenum (FIG. 8). In addition, we also found multiple T2R cDNA sequences in a highly enriched rat gastric endocrine cell cDNA library. All amplified products were cloned and sequenced, confirming that they are identical to known T2R sequences. In contrast, RT-PCR using RNA isolated from liver, submandibular gland, heart, kidney and brain as well as from the non-differentiated intestinal epithelial cell line IEC-6 did not detect any of these transcripts. These results revealed the selective expression of taste receptors of the T2R family in the rat gastric and duodenal mucosa. We also determined if any of the known T2Rs are expressed in mouse gastric and duodenal tissues. RT-PCR and sequencing analysis confirmed that transcripts corresponding to mT2R19 were present in the antrum, fundus and duodenum as well as in the tongue but not in other tissues, including colon, liver, heart and kidney (FIG. 3). However, other GT2Rs originally identified from STC-1 cell including GT2R-S1, -S2, -S7, mT2R5, mT2R8 and mT2R30 were differentially expressed in mouse antrum, fundus and duodenum, although their genes are all located within the bitter locus of chromosome 6 (FIG. 11).

### Example 5

### STC-1 is a New Cell Model for Taste Transduction

Having demonstrated that STC-1 cells express multiple bitter taste receptors as well as α subunits of G proteins that mediate taste signal transduction. We also demonstrated that addition of compounds widely used in bitter taste signaling (e.g., denatonium, phenylthiocarbamide, 6-n-propil-2-thiouracil and cycloheximide) to cultures of STC-1 cells promoted rapid [Ca²⁺]ᵢ responses in these cells (FIG. 9) but not in other well studied cell lines such as IEC-18 or 3T3 (FIG. 10).

Therefore, activation of a single or multiple GT2R promotes the synthesis of second messengers leading to the release of Ca²⁺ from intracellular stores or modulate the gating of ion channels that mediate Ca²⁺ entry into the cell. The increase in [Ca²⁺]ᵢ in response to bitter tastants could trigger the release of signaling molecules that activate neural reflexes and/or modulate the activity of adjacent cells. Given that at the present time there are no long-term cultured cell systems, to study taste receptor-mediated signaling, this invention provides an excellent cell model of STC-1 for studying GT2R gene regulation and GT2R-mediated signal transduction.

### Conclusion

The identification of chemosensory receptors in the stomach and intestine that perceive chemical components of ingested substances including drugs and toxins has a number of important implications including the design of novel molecules that modify responses initiated by activation of these receptors. For example, drugs and toxins initiate vomiting reflexes; several food components regulate appetite and satiety, alter motility of the stomach and intestine and initiate neural and hormonal pathways necessary for normal digestive function. It is likely that the large family of chemosensory receptors that we identified in the stomach and intestine play a major role in mediating these responses.

Taste reception in the post-oral GI tract may be integrated in the central nervous system with taste signals emanating from the lingual epithelium or are processed through an entirely different system. Behavioral effects of bitter compounds (e.g., conditioned taste avoidance) may be the consequence of a complex integration of stimuli, perceived not only at the taste buds but also by taste receptors expressed in the stomach and intestine. The identification of taste receptors in the gastric and duodenal mucosa opens new avenues for understanding molecular sensing and paves the way for developing therapeutic compounds that modify the function of these receptors in the gut.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. An isolated nucleic acid encoding a chemosensing G-protein coupled receptor, wherein the receptor is expressed in a gastroenteric endocrine cell, the receptor constituting greater than 60% nucleic acid sequence identity to a sequence selected from the group consisting of SEQ ID NOS:1, SEQ ID NO:3, SEQ ID NO:5, SEQ ID NO:7, SEQ ID NO:9, SEQ ID NO:11, or SEQ ID NO:13, or from the group consisting of SEQ ID NOS:15, 17, 19, 21, 23, 25, 27, 29, or from the group consisting of SEQ ID NOS:31, 33, 35, 37, 39, 41, 43, 45, 47, 49, 51, 53, 55, 57, 59, 61, 63, 65, 67, or from the group consisting of SEQ ID NOS:69, 71, 73, 75, 77, 79, 81, 83, 85, 87, 89.

2. The isolated nucleic acid of claim 1, wherein the nucleic acid encodes a receptor comprising an amino acid sequence of SEQ ID NO:2, SEQ ID NO:4, SEQ ID NO:6, SEQ ID NO:8, SEQ ID NO:10, SEQ ID NO:12, SEQ ID NO:14.

3. The isolated nucleic acid of claim 1, wherein the nucleic acid comprises a nucleic acid sequence of SEQ ID NO:1, SEQ ID NO:3, SEQ ID NO:5 and SEQ ID NO:7, SEQ ID NO:9, SEQ ID NO:11, or SEQ ID NO:13.

4. The isolated nucleic acid of claim 1, wherein the nucleic acid is from mouse, rat or human origin.

5. The isolated nucleic acid of claim 1, wherein the nucleic acid is amplified by primers that anneal to the same sequence as degenerate primers encoding amino acid sequences selected from the group consisting of SEQ ID NO:2, SEQ ID NO:4, SEQ ID NO:6, SEQ ID NO:8, SEQ ID NO:10, SEQ ID NO:12, SEQ ID NO:14.

6. The method for identifying a compound that modifies chemosensory responses in gastroenteric endocrine cells, the method comprising the steps of:
(i) contacting the compound with a taste-sensing G-protein coupled receptor polypeptide, wherein the polypeptide is expressed in gastroenteric endocrine cell, the polypeptide constituting greater than 50% amino acid sequence identity to a sequence selected from the group consisting of (a) mouse GT2R: SEQ ID NOS:2, 4, 6, 8, 10, 12, 14, 16, 18, 20, 22, 24, 26, 28, 30, or from the group consisting of (b) rat GT2R: SEQ ID NOS: 32, 34, 36, 38, 40, 42, 44, 46, 48, 50, 52, 54, 56, 58, 60, 62, 64, 66, 68, or from the group consisting of (c) human GT2R SEQ ID NOS:70, 72, 74, 76, 78, 80, 82, 84, 86, 88, 90.
(ii) determining the functional effect of the compound on the polypeptide.

7. The method of claim 6, wherein the polypeptide has G-protein coupled receptor activity.

8. The method of claim 6, wherein the functional effect is determined by measuring changes in intracellular Ca²⁺, cyclic nucleotides, phosphorylation and dephosphorylation, and pH.

9. The method of claim 6, wherein the functional effect is determined by measuring peptide hormone and neurotransmitter release.

10. The method of claim 6, wherein the polypeptide is native or recombinant.

11. The method of claim 6, wherein the polypeptide is from mouse, rat, and human origin.

12. The method of claim 6, wherein the polypeptide is expressed in gastrointestinal cells including but not exclusively endocrine and exocrine cells, epithelial cells and neuroendocrine cells.

13. The method of claim 6, wherein the functional effect is determined by measuring changes in receptor phosphorylation, internalization, and redistribution.

14. The method of claim 6, wherein the cell is a eukaryotic cell.

15. The method of claim 6, wherein the polypeptide comprises an amino acid sequence selected from the group consisting of (a) mouse GT2R: SEQ ID NOS:2, 4, 6, 8, 10, 12, 14, 16, 18, 20, 22, 24, 26, 28, 30, or from the group consisting of (b) rat GT2R: SEQ ID NOS: 32, 34, 36, 38, 40, 42, 44, 46, 48, 50, 52, 54, 56, 58, 60, 62, 64, 66, 68, or from the group consisting of (c) human GT2R SEQ ID NOS:70, 72, 74, 76, 78, 80, 82, 84, 86, 88, 90.

16. An expression vector comprising the nucleic acid of claim 1.

17. An isolated cell comprising the vector of claim 16.

18. The use of native STC-1 enteroendocrine cells that naturally express GT2R to identify modulators of taste receptor-mediated signal transduction.

19. The method of claim 18 to identify modulators of GT2R-mediated signal transduction, the method comprising the step of:
contacting a STC-1 enteroendocrine cell with a compound
measuring functional effect of the compound on STC-1 cells

20. The method of claim 18, wherein the modulators are taste molecules from food or pharmaceutical components, their breakdown products, or contaminants.

21. The method of claim 18, wherein the signal transduction is determined by measuring changes in intracellular Ca²⁺, cyclic nucleotides, phosphorylation and dephosphorylation, pH, and cholecystokinin (CCK) release.
